(19) United States
(12) Patent Application Publication
(10) Pub. No.: US 2012/0107413 A1
(43) Pub. Date: May 3, 2012

(54) PRE-NATAL MESENCHYMAL STEM CELLS

(75) Inventors: Sai Kiang Lim, Singapore (SG); Kok Hian Tan, Singapore (SG)

(73) Assignees: SINGAPORE HEALTH SERVICES PTE LTD, Singapore (SG); AGENCY FOR SCIENCE, TECHNOLOGY AND RESEARCH, Singapore (SG)

(21) Appl. No.: 13/383,294

(22) PCT Filed: Jul. 21, 2010
(86) PCT No.: PCT/SG2010/000275

§ 371 Date: Jan. 10, 2012

Related U.S. Application Data
(60) Provisional application No. 61/227,865, filed on Jul. 23, 2009.

(51) Int. Cl. A61K 35/54; A61P 9/10; C12N 1/00; C12N 5/07; C12N 5/0775

(52) U.S. Cl. 424/582; 435/325; 435/366; 435/317.1

(57) ABSTRACT

We describe an pre-natal mesenchymal stem cell obtainable from a pre-natal tissue such as a foetal tissue, a descendent of such a mesenchymal stem cell, a cell culture or a cell line comprising either. The pre-natal mesenchymal stem cell, cell culture or cell line may comprise a cell line F1Ib, F2lb, F3lb, F1ki or F3li. We further describe a conditioned medium conditioned by such a pre-natal mesenchymal stem cell, cell culture or cell line. These may comprise cardioprotective activity, and may in particular be used to treat or prevent a range of cardiac disorders of diseases.

### FIELD

The present invention relates to the fields of development, cell biology, molecular biology and genetics. More particularly, the invention relates to a method of deriving mesenchymal stem cells from fetal stem cells.

### BACKGROUND

Stem cells, unlike differentiated cells have the capacity to divide and either self-renew or differentiate into phenotypically and functionally different daughter cells (Keller, Genes Dev. 2005; 19:1129-1155; Wobus and Boheler, Physiol Rev. 2005; 85:635-678; Wiles, Methods in Enzymology. 1993; 225:900-918; Choi et al, Methods Mol Med. 2005; 105:359-368).

Mesenchymal stem cells (MSCs) are multipotent stem cells that have a limited but robust potential to differentiate into mesenchymal cell types, e.g. adipocytes, chondrocytes and osteocytes, with negligible risk of teratoma formation.

MSC transplantation has been used to treat musculoskeletal injuries, improve cardiac function in cardiovascular disease and ameliorate the severity of graft-versus-host-disease¹. Most MSC transplantations are either autologous or immune-compatible allogeneic transplantations, since MSCs can be easily harvested from accessible adult tissues such as bone marrow, adipose tissues, cord blood and expanded ex vivo. Therefore, host immune rejection of transplanted MSCs is easily circumvented.

In recent years, MSC transplantations have demonstrated therapeutic efficacy in treating different diseases but the underlying mechanism has been controversial²⁻¹⁰. Some reports have suggested that factors secreted by MSCs¹¹ were responsible for the therapeutic effect on arteriogenesis¹², stem cell crypt in the intestine¹³, ischemic injury^{10, 14-19}, and hematopoiesis^{20,21}.

More recently, it was demonstrated that intramyocardial administration of cultured media conditioned by rat Akt-transformed BM-MSCs reduces ventricular remodeling and improves cardiac function in a rodent model of myocardial ischemia¹⁰. Secretion from untransformed rat BM-MSCs, however, was not cardioprotective¹⁷. It was subsequently demonstrated that the major mediator of cardioprotection in the secretion of Akt-transformed BM-MSCs was Sfp2 whose expression was a direct consequence of Akt overexpression and the resulting upregulation of the PI3K pathway²².

These observations suggest that rodent BM-MSCs do not produce cardioprotective secretion but could be modified to produce cardioprotective secretion. Others have also reported similar observations²³

### SUMMARY

We recently demonstrated that human MSCs derived from human embryonic stem cells (hESC-MSCs)²⁴ secrete >200 proteins²⁵ and that a bolus administration of hESC-MSCs conditioned medium (CM) 5 minutes prior to reperfusion significantly reduced infarct size by 60% and improved cardiac function in a pig and mouse model of myocardial ischemia-reperfusion (MI/R) injury²⁶.

The present invention is based on the demonstration that the different developmental stage from which MSCs are derived i.e. embryonic versus adult, is important for the production of cardioprotective secretion. We demonstrate that secretion of MSCs derived directly from another non-adult tissue, such as fetal tissue, is similarly cardioprotective.

The Examples show the generation of five MSC cultures, F1lb, F1ki, F2lb, F3lb and F3i from limb (lb), kidney (ki) and liver (li) tissues of three fetuses in three independent experiments. The Examples demonstrate that these fetal MSCs fulfil the defining criteria of a MSC. The Examples also show that these fetal MSCs was highly proliferative and their secretion reduced infarct size in a mouse model of ischemia/reperfusion injury.

According to a 1^{st} aspect of the present invention, we provide a pre-natal mesenchymal stem cell. The pre-natal mesenchymal stem cell may be obtainable from a pre-natal tissue such as a foetal tissue. We further provide a descendent of such a mesenchymal stem cell, a cell culture or a cell line comprising either.

The pre-natal mesenchymal stem cell, cell culture or cell line may comprise a cell line F1lb. The pre-natal mesenchymal stem cell, cell culture or cell line may comprise a cell line F2lb, a cell line F3lb, a cell line F1ki or a cell line F3li.

The pre-natal mesenchymal stem cell, cell culture or cell line may comprise cardioprotective activity. The cardioprotective activity may be, assayed in a mouse model of acute myocardial infarction (AMI).

The pre-natal mesenchymal stem cell, cell culture or cell line may be obtainable by a method which comprises providing a pre-natal tissue. The pre-natal tissue may be contacted with a plastic surface. The mesenchymal stem cells comprised in the pre-natal tissue may be allowed to adhere to the plastic surface.

The method may further comprise culturing the mesenchymal stem cell in a serum free medium. The serum free medium may comprise serum-free culture medium such as Knockout DMEM medium. This may be supplemented with 10% serum replacement media. It may be supplemented with non-essential amino acids. It may be supplemented with 10 ng/ml FGF2. It may be supplemented with 10 ng/ml Recombinant Human EGF. It may be supplemented with 55 μM β-mercaptoethanol.

The pre-natal mesenchymal stem cell, cell culture or cell line may be such that the pre-natal mesenchymal stem cell displays one or more of the following characteristics. The pre-natal mesenchymal stem cell may display one or more morphological characteristics of mesenchymal stem cells. Such a characteristic may include fingerprint whorl at confluency. It may include forming an adherent monolayer with a fibroblastic phenotype.

The pre-natal mesenchymal stem cell, cell culture or cell line may be capable of adhering to plastic. The pre-natal mesenchymal stem cell, cell culture or cell line may display an average population doubling time of between 72 to 96 hours. The pre-natal mesenchymal stem cell, cell culture or cell line may display a surface antigen profile comprising expression of one or more, such as all, of the following: CD29, CD44, CD49a, CD49e, CD105, CD166, MHC I. It may display a reduced or absent expression of one or more of the following: HLA-DR, CD34 and CD45. The pre-natal mesenchymal stem cell, cell culture or cell line may be CD29+, CD44+, CD49a+ CD49e+, CD105+, CD166+, MHC I+, CD34⁻ and CD45⁻.

The pre-natal mesenchymal stem cell, cell culture or cell line may display a reduced expression of one or more, such as all, of HESX1, POUFL5, SOX-2, UTF-1 and ZFP42. The pre-natal mesenchymal stem cell, cell culture or cell line may display a reduced expression of one or more, such as all, of OCT4, NANOG and SOX2. The pre-natal mesenchymal stem cell, cell culture or cell line may display no detectable alkaline phosphatase activity.

The pre-natal mesenchymal stem cell, cell culture or cell line may be maintainable in cell culture for greater than 10, 20, 30, 40 or more generations. The pre-natal mesenchymal stem cell, cell culture or cell line may have a substantially stable karyotype or chromosome number when maintained in cell culture for at least 10 generations. The pre-natal mesenchymal stem cell, cell culture or cell line may be such that it does not substantially induce formation of teratoma when transplanted to a recipient animal. The recipient animal may comprise an immune compromised recipient animal. The time period may be after 3 weeks, such as after 2 to 9 months. The pre-natal mesenchymal stem cell, cell culture or cell line may be such that it not teratogenic when implanted in SCID mice.

The pre-natal mesenchymal stem cell, cell culture or cell line may be such that it is negative for mouse-specific c-mos repeat sequences and positive for human specific alu repeat sequences. The pre-natal mesenchymal stem cell, cell culture or cell line may be capable of undergoing osteogenesis, adipogenesis or chondrogenesis, such as capable of differentiating into osteocytes, adipocytes or chondrocytes. The pre-natal mesenchymal stem cell, cell culture or cell line may have a substantially stable gene expression pattern from generation to generation.

The pre-natal mesenchymal stem cell, cell culture or cell line may be such that any two or more, such as all, mesenchymal stem cells obtainable by the method exhibit substantially identical gene expression profiles. It may be such that the gene expression correlation coefficient between any two or more isolates of mesenchymal stem cells obtained by the method is greater than 0.9. It may be such that any two or more, such as all, isolates of mesenchymal stem cells obtainable by the method are substantially similar or identical (such as homogenous) with each other. It may be such that the gene expression correlation coefficient between a mesenchymal stem cell obtainable by the method and cells of a parental culture is greater than 0.8.

The pre-natal mesenchymal stem cell, cell culture or cell line may comprise a mammalian, such as a mouse or human, pre-natal mesenchymal stem cell, cell culture or cell line.

There is provided, according to a 2^{nd} aspect of the present invention, a conditioned medium comprising medium conditioned by a pre-natal mesenchymal stem cell, cell culture or cell line as described above.

We provide, according to a 3^{rd} aspect of the present invention, a particle secreted by a pre-natal mesenchymal stem cell as described above and comprising at least one biological property of a pre-natal mesenchymal stem cell such as a biological activity of a pre-natal mesenchymal stem cell conditioned medium (MSC-CM), for example cardioprotection.

As a 4^{th} aspect of the present invention, there is provided a pre-natal mesenchymal stem cell, cell culture, cell line, conditioned medium or particle as set out above for use in a method of treatment of a disease. The disease may be selected from the group consisting of: cardiac failure, bone marrow disease, skin disease, burns and degenerative diseases such as diabetes, Alzheimer's disease, Parkinson's disease, cancer, a disease associated with accumulation of protein aggregates or intracellular or extracellular lesions; Huntingdon's disease and alcoholic liver disease.

The disease may be selected from the group consisting of: myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

The pre-natal mesenchymal stem cell, cell culture, cell line, conditioned medium or particle may be used to aid wound healing, scar reduction, bone formation, a bone graft or bone marrow transplantation.

The pre-natal mesenchymal stem cell, cell culture, cell line, conditioned medium or particle may be used to regulate or assist in the regulation of a pathway selected from any one or more of the following: cytoskeletal regulation by Rho GTPase, cell cycle, integrin signalling pathway, Inflammation mediated by chemokine & cytokine signaling pathway, FGF signaling pathway, EGF receptor signaling pathway, angiogenesis, plasminogen activating cascade, blood coagulation, glycolysis, ubiquitin proteasome pathway, de novo purine biosynthesis, TCA cycle, phenylalanine biosynthesis, heme biosynthesis.

The pre-natal mesenchymal stem cell, cell culture, cell line, conditioned medium or particle may be used in the regulation of processes including any one or more of the following: cell structure and motility, cell structure, cell communication, cell motility, cell adhesion, endocytosis, mitosis, exocytosis, cytokinesis, cell cycle, immunity and defense, cytokine/chemokine mediated immunity, macrophage-mediated immunity, granulocyte-mediated immunity, ligand-mediated signaling, cytokine and chemokine mediated signaling pathway, signal transduction, extracellular matrix protein-mediated signaling, growth factor homeostasis, receptor protein tyrosine kinase signaling pathway, cell adhesion-mediated signaling, cell surface receptor mediated signal transduction, JAK-STAT cascade, antioxidation and free radical removal, homeostasis, stress response, blood clotting, developmental processes, mesoderm development, skeletal development, angiogenesis, muscle development, muscle contraction, protein metabolism and modification, proteolysis, protein folding, protein complex assembly, amino acid activation, intracellular protein traffic, other protein targeting and localization, amino acid metabolism, protein biosynthesis, protein disulfide-isomerase reaction, carbohydrate metabolism, glycolysis, pentose-phosphate shunt, other polysaccharide metabolism, purine metabolism, regulation of phosphate metabolism, vitamin metabolism, amino acid biosynthesis, pre-mRNA processing, translational regulation, mRNA splicing.

The pre-natal mesenchymal stem cell, cell culture, cell line, conditioned medium or particle may be used in the supply of functions including any one or more of the following: signaling molecule, chemokine, growth factor, cytokine, interleukin, other cytokine, extracellular matrix, extracellular matrix structural protein, other extracellular matrix, extracellular matrix glycoprotein, protease, metalloprotease, other proteases, protease inhibitor, metalloprotease inhibitor, serine protease inhibitor, oxidoreductase, dehydrogenase, peroxidase, chaperone, chaperonin, Hsp 70 family chaperone, other chaperones, synthetase, synthase and synthetase, select calcium binding protein, aminoacyl-tRNA synthetase, lyase, isomerase, other isomerase, ATP synthase, hydratase, transaminase, other lyase, other enzyme regulator, select regulatory molecule, actin binding cytoskeletal protein, cytoskeletal protein, non-motor actin binding protein, actin and actin related protein, annexin, tubulin, cell adhesion molecule, actin binding motor protein, intermediate filament, ribonucleoprotein, ribosomal protein, translation factor, other RNA-binding protein, histone, calmodulin related protein, vesicle coat protein.

We provide, according to a 5^{th} aspect of the present invention, a delivery system for delivering a conditioned medium or particle, comprising a source of conditioned medium or particle as described above together with a dispenser operable to deliver the conditioned medium or particle to a target.

The present invention, in a 6^{th} aspect, provides for the use of such a delivery system in a method of delivering a conditioned medium or particle to a target.

In a 7^{th} aspect of the present invention, there is provided a method of obtaining a differentiated mesenchymal cell, the method comprising providing a pre-natal mesenchymal stem cell, cell culture or cell line as described above and differentiating the pre-natal mesenchymal stem cell, cell culture or cell line into an osteocyte, an adipocyte or a chondrocyte.

According to an 8^{th} aspect of the present invention, we provide a differentiated mesenchymal cell obtainable by such a method.

We provide, according to a 9^{th} aspect of the invention, a pharmaceutical composition comprising a pre-natal mesenchymal stem cell, cell culture, cell line, conditioned medium or particle as set out above or a conditioned medium as described, together with a pharmaceutically acceptable excipient or carrier.

There is provided, in accordance with a 10^{th} aspect of the present invention, a method of conditioning a cell culture medium, the method comprising culturing a pre-natal mesenchymal stem cell, cell culture or cell line as described in a cell culture medium and optionally isolating the cell culture medium.

As an 11^{th} aspect of the invention, we provide a method of treatment of a disease comprising obtaining a pre-natal mesenchymal stem cell, cell culture, cell line, conditioned medium or particle as set out above and administering the pre-natal mesenchymal stem cell, cell culture, cell line, conditioned medium or particle into a patient.

The disease may be selected from the diseases set out in the 3^{rd} aspect of the invention.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-3,4-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 is a diagram showing characterisation of fetal MSC cultures.

FIG. 1A (left hand panels). Cellular morphology under phase contrast. Representative images of the five different MSCs, F1lb (p8) and F1ki (p8) derived from the limb and kidney tissues of the same fetus; F2lb (p8) derived from the limb of a second fetus; and F3lb (p8) and F3li (p8) derived from the limb and liver tissues of the same third fetus.

FIG. 1B (right hand panels). Karyotype analysis by G-banding was performed each of the fetal MSC cultures, F1lb (p10) F2lb (p10), F3lb (p10), F1ki (p12), and F1li (p12).

FIG. 2 is a diagram showing telomerase activity in hESC-MSCs and fetal MSCs. Relative telomerase activity was measured by real time quantitative telomeric repeat amplification protocol. This qPCR-based assay quantifies product generated in vitro by telomerase activity present in the samples. The relative telomerase activity which is directly proportional to the amount of telomerase products was assessed by the threshold cycle number (or Ct value) for one ug protein cell lysate. Hues9.E1 referred to a previously described hESC-MSCs line and HEK is a human embryonic kidney cell line. The Ct value for each fetal MSCs was the mean for three passages, P16, P18, and P20, and that for Hues9.E1 was the mean for two passages, P20 and P22. The assay was performed in triplicate for each passage.

FIG. 3 is a diagram showing marker profiling. (A, B) F1lb MSCs at p11 or p12 were stained with a specific antibody conjugated to a fluorescent dye and analyzed by FACS. Nonspecific fluorescence was determined by incubation of similar cell aliquots with isotype-matched mouse monoclonal antibodies. (C) Relative transcription level of OCT4 and SOX2 were measured using quantitative RT-PCR. hES3, a human embryonic stem cells line was set as the baseline for comparison.

FIG. 4 is a diagram showing differentiation of fetal MSCs. Fetal MSCs were induced to undergo osteogenesis, adipogenesis and chondrogenesis. After osteogenesis (FIG. 4A), adipogenesis (FIG. 4B) and chondrogenesis (FIG. 4C), the differentiated cells were stained with von Kossa stain, Oil Red and Alcian blue, respectively. Images of differentiated fetal MSCs as represented by differentiated F3lb MSCs at 100× magnification.

FIG. 5 is a diagram showing gene expression analysis. Total RNA was prepared in technical replicates from different passages of F1lb (p10, p12, p14), F1ki (p10, p14, p16), F2lb (p10, p12, p14), F3lb (p10, p12, p14) and F3li (p10, p16), and from two technical replicates of the previously described hESC-MSCs line, Hues9.E1 (p19). 750 ng of biotinylated cRNA from each sample were used for microarray analysis on the Sentrix HumanRef-8 Expression BeadChip Version 3 (Illumina, Inc., San Diego, Calif.). The gene expression profile of all samples were normalized by a shift to the 75th percentile, baseline transformed to median of all samples, and a heatmap of correlation between pairs of array plotted.

FIG. 6 is a diagram showing cardioprotective secretion

FIG. 6A. Proteins in culture medium conditioned by hESC-MSCs (Hues9.E1), F1lb, F1ki or F3lb was separated on a 4-12% SDS-PAGE gradient gel and stained with silver. Two proteins were loaded in each lane.

FIG. 6B. Western blot analysis of the conditioned medium described in (A). The amount of proteins loaded were 4, 16, 16 and 16 μg, respectively.

FIG. 6C. Infarct size (IS) as a percentage of the area at risk (AAR) upon treatment with saline (n=10), conditioned medium from hESC-MSCs (n=10), F1lb-MSCs (n=6) and F1ki-MSCs (n=6). Saline treatment resulted in 34.5±3.3% infarction, whereas conditioned medium from hESC-MSCs, F1lb-MSCs and F1ki-MSCs resulted in 21.2±3.3%, 17.4±3.7% and 19.9±2.6%, respectively.

FIG. 6D. AAR as a percentage of the left ventricle (LV), showing the amount of endangered myocardium after MI/R injury. All animals were affected to the same extent by the operative procedure, resulting in 39.4±2.0% of AAR among the groups. Each bar represents Mean±SEM.

FIG. 7 is a diagram showing cardioprotective HPLC-isolated microparticles.

FIG. 7A. HPLC fractionation and dynamic light scattering of F1lb CM and NCM. F1lb CM and NCM were fractionated on a HPLC using BioSep S4000, 7.8 mm×30 cm column. The components in F1lb CM or NCM were eluted with 150 mM of NaCl in 20 mM phosphate buffer, pH 7.2. The elution mode was isocratic and the run time was 40 minutes. The eluent was monitored with an UV-visible detector set at 220 ηm and light scattering signal was collected. The solid rhombus represented light scattering signal as measured in voltage.

FIG. 7B. The eluted fractions, F1 to F12 were collected, their volumes were adjusted to 10% of the input volume of CM and equal volume of F1-F12 were separated by gel electrophoresis and stained with silver.

FIG. 7C. Infarct size (IS) as a percentage of the area at risk (AAR) upon treatment with saline (n=10), F1lb CM (n=6) and HPLC F1 (n=6). Saline-treated mice had a 34.5±3.3% relative infarct size while F1lb CM- and HPLC F1-treated mice had a 17.4±3.7% and 18.1±2.0% relative infarct size, respectively.

FIG. 7D. AAR as a percentage of the left ventricle (LV), showing the amount of endangered myocardium after MI/R injury. All animals were affected to the same extent by the operative procedure, resulting in 39.4±2.0% of AAR among the groups. Each bar represents Mean±SEM.

### DETAILED DESCRIPTION

### Pre-Natal Mesenchymal Stem Cells

We describe a mesenchymal stem cell which is derivable from a pre-natal cell. Such a mesenchymal stem cell may be referred to in this document as a “pre-natal mesenchymal stem cell” or a mesenchymal stem cell obtained by the methods described in this document.

The pre-natal cell from which the mesenchymal stem cell is derived may comprise a foetal cell, such as a cadaveric foetal cell. The foetal cell may comprise a first or second trimester foetal cell. The foetal cell may comprise a cell of any suitable age, for example up to 2, 4, 8, 16 or 24 weeks.

The foetal cell may comprise a cell from any tissue such as a limb cell, a kidney cell or a liver cell. The pre-natal cell from which the mesenchymal stem cell is derived may be comprised in a tissue. The tissue may be derived from a cadaveric foetus. The mesenchymal stem cell may comprise a mammalian, primate or human mesenchymal stem cell.

The mesenchymal stem cell line may comprise an F1lb, F2lb, F3lb, F1ki or F3li cell line.

We further provide a medium which is conditioned by culture of the pre-natal mesenchymal stem cells. Such a conditioned medium is referred to in this document as a “pre-natal mesenchymal stem cell conditioned medium” and is described in further detail below.

We further provide a particle secreted by a pre-natal mesenchymal stem cell and comprising at least one biological property of a pre-natal mesenchymal stem cell. We refer to such a particle in this document as a “pre-natal mesenchymal stem cell particle”. Such a particle is described in further detail below, and a summary follows.

The biological property may comprise a biological activity of a pre-natal mesenchymal stem cell conditioned medium (MSC-CM). The biological activity may comprise cardioprotection. The pre-natal mesenchymal stem cell particle may be capable of reducing infarct size.

Reduction of infarct may be assayed in a mouse or pig model of myocardial ischemia and reperfusion injury.

The pre-natal mesenchymal stem cell particle may be capable of reducing oxidative stress. The reduction of oxidative stress may be assayed in an in vitro assay of hydrogen peroxide (H₂O₂)-induced cell death.

The pre-natal mesenchymal stem cell particle comprise a vesicle. The pre-natal mesenchymal stem cell particle may comprise an exosome.

The pre-natal mesenchymal stem cell particle may contain at least 70% of proteins in an pre-natal mesenchymal stem cell conditioned medium (MSC-CM).

The pre-natal mesenchymal stem cell particle may comprise a complex of molecular weight >100 kDa. The complex of molecular weight >100 kDa may comprise proteins of <100 kDa. The particle may comprise a complex of molecular weight >300 kDa. The complex of molecular weight >100 kDa may comprise proteins of <300 kDa.

The pre-natal mesenchymal stem cell particle may comprise a complex of molecular weight >1000 kDa. The particle may have a size of between 2 nm and 200 nm. The pre-natal mesenchymal stem cell particle may have a size of between 50 ηm and 150 nm. The pre-natal mesenchymal stem cell particle may have a size of between between 50 nm and 100 nm.

The size of the pre-natal mesenchymal stem cell particle may be determined by filtration against a 0.2 μM filter and concentration against a membrane with a molecular weight cut-off of 10 kDa. The size of the pre-natal mesenchymal stem cell particle may be determined by electron microscopy.

The pre-natal mesenchymal stem cell particle may comprise a hydrodynamic radius of below 100 nm. It may comprise a hydrodynamic radius of between about 30 nm and about 70 nm. It may be between about 40 nm and about 60 nm, such as between about 45 nm and about 55 nm. The pre-natal mesenchymal stem cell particle may comprise a hydrodynamic radius of about 50 nm. The hydrodynamic radius may be determined by laser diffraction or dynamic light scattering.

The pre-natal mesenchymal stem cell particle may comprise a lipid selected from the group consisting of: phospholipid, phosphatidyl serine, phosphatidyl inositol, phosphatidyl choline, shingomyelin, ceramides, glycolipid, cerebroside, steroids, cholesterol. The cholesterol-phospholipid ratio may be greater than 0.3-0.4 (mol/mol). The pre-natal mesenchymal stem cell particle may comprise a lipid raft.

The pre-natal mesenchymal stem cell particle may be insoluble in non-ionic detergent, preferably Triton-X100. The pre-natal mesenchymal stem cell particle may be such that proteins of the molecular weights specified substantially remain in the complexes of the molecular weights specified, when the pre-natal mesenchymal stem cell particle is treated with a non-ionic detergent.

The pre-natal mesenchymal stem cell particle may be sensitive to cyclodextrin, preferably 20 mM cyclodextrin. The pre-natal mesenchymal stem cell particle may be such that treatment with cyclodextrin causes substantial dissolution of the complexes specified.

The pre-natal mesenchymal stem cell particle may comprise ribonucleic acid (RNA). The particle may have an absorbance ratio of 1.9 (260:280 nm). The pre-natal mesenchymal stem cell particle may comprise a surface antigen selected from the group consisting of: CD9, CD109 and thy-1.

We further describe a method of producing a pre-natal mesenchymal stem cell particle as described above, the method comprising isolating the pre-natal mesenchymal stem cell particle from a pre-natal mesenchymal stem cell conditioned medium (MSC-CM).

The method may comprise separating the pre-natal mesenchymal stem cell particle from other components based on molecular weight, size, shape, composition or biological activity.

The weight may be selected from the weights set out above. The size may be selected from the sizes set out above. The composition may be selected from the compositions set out above. The biological activity may be selected from the biological activities set out above.

We further describe a method of producing a pre-natal mesenchymal stem cell particle as described above. The method may comprise obtaining a pre-natal mesenchymal stem cell conditioned medium (MSC-CM). It may comprise concentrating the pre-natal mesenchymal stem cell conditioned medium. The pre-natal mesenchymal stem cell conditioned medium may be concentrated by ultrafiltration over a >1000 kDa membrane. The method may comprise subjecting the concentrated pre-natal mesenchymal stem cell conditioned medium to size exclusion chromatography. A TSK Guard column SWXL, 6×40 mm or a TSK gel G4000 SWXL, 7.8×300 mm column may be employed. The method may comprise selecting UV absorbent fractions, for example, at 220 nm, that exhibit dynamic light scattering. The dynamic light scattering may be detected by a quasi-elastic light scattering (QELS) detector. The method may comprise collecting fractions which elute with a retention time of 11-13 minutes, such as 12 minutes.

We further provide a pharmaceutical composition comprising a pre-natal mesenchymal stem cell particle as described together with a pharmaceutically acceptable excipient, diluent or carrier.

We further provide such a pre-natal mesenchymal stem cell particle or such a pharmaceutical composition for use in a method of treating a disease.

We further provide for the use of such a pre-natal mesenchymal stem cell particle for the preparation of a pharmaceutical composition for the treatment of a disease.

We further provide for the use of such a pre-natal mesenchymal stem cell particle in a method of treatment of a disease in an individual.

The disease may be selected from the group consisting of: cardiac failure, bone marrow disease, skin disease, burns and degenerative diseases such as diabetes, Alzheimer's disease, Parkinson's disease and cancer.

The disease may be selected from the group consisting of: myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

The pre-natal mesenchymal stem cell particle may be used to aid wound healing, scar reduction, bone formation, a bone graft or bone marrow transplantation in an individual.

The pre-natal mesenchymal stem cell particle may be used (i) in the regulation of a pathway selected from any one or more of the following: cytoskeletal regulation by Rho GTPase, cell cycle, integrin signalling pathway, Inflammation mediated by chemokine & cytokine signaling pathway, FGF signaling pathway, EGF receptor signaling pathway, angiogenesis, plasminogen activating cascade, blood coagulation, glycolysis, ubiquitin proteasome pathway, de novo purine biosynthesis, TCA cycle, phenylalanine biosynthesis, heme biosynthesis; (ii) in the regulation of processes including any one or more of the following: cell structure and motility, cell structure, cell communication, cell motility, cell adhesion, endocytosis, mitosis, exocytosis, cytokinesis, cell cycle, immunity and defense, cytokine/chemokine mediated immunity, macrophage-mediated immunity, granulocyte-mediated immunity, ligand-mediated signaling, cytokine and chemokine mediated signaling pathway, signal transduction, extracellular matrix protein-mediated signaling, growth factor homeostasis, receptor protein tyrosine kinase signaling pathway, cell adhesion-mediated signaling, cell surface receptor mediated signal transduction, JAK-STAT cascade, antioxidation and free radical removal, homeostasis, stress response, blood clotting, developmental processes, mesoderm development, skeletal development, angiogenesis, muscle development, muscle contraction, protein metabolism and modification, proteolysis, protein folding, protein complex assembly, amino acid activation, intracellular protein traffic, other protein targeting and localization, amino acid metabolism, protein biosynthesis, protein disulfide-isomerase reaction, carbohydrate metabolism, glycolysis, pentose-phosphate shunt, other polysaccharide metabolism, purine metabolism, regulation of phosphate metabolism, vitamin metabolism, amino acid biosynthesis, pre-mRNA processing, translational regulation, mRNA splicing; or (iii) in the supply of functions including any one or more of the following: signaling molecule, chemokine, growth factor, cytokine, interleukin, other cytokine, extracellular matrix, extracellular matrix structural protein, other extracellular matrix, extracellular matrix glycoprotein, protease, metalloprotease, other proteases, protease inhibitor, metalloprotease inhibitor, serine protease inhibitor, oxidoreductase, dehydrogenase, peroxidase, chaperone, chaperonin, Hsp 70 family chaperone, other chaperones, synthetase, synthase and synthetase, select calcium binding protein, aminoacyl-tRNA synthetase, lyase, isomerase, other isomerase, ATP synthase, hydratase, transaminase, other lyase, other enzyme regulator, select regulatory molecule, actin binding cytoskeletal protein, cytoskeletal protein, non-motor actin binding protein, actin and actin related protein, annexin, tubulin, cell adhesion molecule, actin binding motor protein, intermediate filament, ribonucleoprotein, ribosomal protein, translation factor, other RNA-binding protein, histone, calmodulin related protein, vesicle coat protein.

We further provide for a delivery system for delivering a pre-natal mesenchymal stem cell particle, comprising a source of pre-natal mesenchymal stem cell particle together with a dispenser operable to deliver the particle to a target.

We further provide use of such a delivery system in a method of delivering a particle to a target.

We demonstrate in the Examples that pre-natal mesenchymal stem cells mediate cardioprotective effects through secreted large complexes of ˜50-100 nm in diameter. Such complexes or particles may therefore be used for therapeutic means, including for cardioprotection, in place of the cells themselves.

The pre-natal mesenchymal stem cell particles, complexes or exosomes may be used for a variety of purposes, such as treatment or prevention for cardiac or heart diseases such as ischaemia, cardiac inflammation or heart failure. They may also be used for repair following perfusion injury.

### Derivation of Pre-Natal Mesenchymal Stem Cells

Pre-natal mesenchymal stem cells as described in this document may be obtained in a number of ways. For example, they may be derived from pre-natal tissue. The pre-natal tissue, such as a foetal tissue, may be processed, such as by dissection, mincing or washing, or any combination thereof.

The mesenchymal stem cell may be derived from such a tissue based on any suitable property, such as preferential adhesion. For example, the mesenchymal stem cell may be selected based on its ability to adhere to a substrate. The substrate may for example comprise plastic. Accordingly, the mesenchymal stem cell may be derived from pre-natal tissue by allowing the mesenchymal stem cells in the pre-natal tissue mass to adhere to plastic. For this purpose, the tissue may be placed on a vessel with one or more plastic surfaces. Such a vessel may comprise a culture vessel, for example a tissue culture plate. The vessel may be gelatinised. The mesenchymal stem cells may be allowed to migrate out of the tissue mass. They may be allowed to adhere to the surface of the vessel. The rest of the pre-natal tissue may be removed, such as by washing off. Thus, the bulk of the tissue pieces may then be washed off, leaving a homogenous cell culture.

The tissue may be cultured in any suitable medium, such as Dulbecco's Modified Eagle Medium. The medium may be supplemented with any suitable supplement, such as serum replacement medium, EGF, FGF2, etc. The serum replacement medium, where present, may be included at any suitable concentration such as 10%. EGF, where present, may be included at any suitable concentration such as 5, 10, 15 or 20 ng/ml. FGF2, where present, may be included at any suitable concentration such as 5, 10, 15 or 20 ng/ml.

A specific protocol which may be used for deriving pre-natal mesenchymal stem cells may comprise the following. The cultures may be cultured in either serum-free or serum-containing culture medium. When confluent, the cultures may be passaged for example by trypsinizing and then splitting such as at 1:4 on a gelatinized tissue culture plate. Serum-free culture medium may be made up of Knockout DMEM medium supplemented with 10% serum replacement media, non-essential amino acids, 10 ng/ml FGF2, 10 ng/ml Recombinant Human EGF and 55 μM β-mercaptoethanol. Serum-containing culture medium may be made up of DMEM-high glucose without glutamine supplemented with penicillin-streptomycin, L-glutamine, non-essential amino acids and 10% fetal calf serum. Instead of, or in addition to EGF, PDGF may be used.

The culture, may comprise or be established in the absence of co-culture, such as in the absence of feeder cells. For this purpose, the mesenchymal stem cell may be cultured without a feeder cell layer. This is described in further detail below.

The mesenchymal stem cell may be derived by optionally selecting a mesenchymal stem cell from other cells based on expression of a cell surface marker, as described in further detail below. The cell may therefore optionally be selected by detecting elevated expression of for example CD105 (Accession Number NM_{—}000118.1) or CD73 (Accession Number NM_{—}002526.1), or both. The cell may be further optionally selected by detecting a reduced expression of CD24 (Accession Number NM_{—}013230.1). Thus, the mesenchymal stem cell may be obtained by selecting for cells which are CD105+CD24−. The mesenchymal stem cell may be selected by labelling the cell with an antibody against the appropriate surface antigen and may be selected by fluorescence activated cell sorting (FACS) or magnetic cell sorting (MACS).

### Characteristics of Pre-Natal Mesenchymal Stem Cells

The mesenchymal stem cells obtained by the methods and compositions described here may display one or more properties or characteristics of mesenchymal stem cells.

They may satisfy any one or more of the morphologic, phenotypic and functional criteria commonly used to identify mesenchymal stem cells⁹, as known in the art. The properties or characteristics may be as defined by The International Society for Cellular Therapy. In particular, they may display one or more characteristics as set out in Dominici et al (2006).

Morphology

The mesenchymal stem cells obtained by the methods and compositions described here may exhibit one or more morphological characteristics of mesenchymal stem cells.

The mesenchymal stem cells obtained by the methods described here may display a typical fingerprint whorl at confluency.

The mesenchymal stem cells obtained may form an adherent monolayer with a fibroblastic phenotype. The mesenchymal stem cell may be capable of adhering to plastic.

They may have an average population doubling time of between 72 to 96 hours. The optimal culture may be at 25% to 85% confluency or 15-50,000 cells per cm².

Antigen Profile

Furthermore, the mesenchymal stem cells obtained may display a surface antigen profile which is similar or identical to mesenchymal stem cells.

The mesenchymal stem cells obtained by the methods described here may lack or display reduced expression of one or more pluripotency marker, such as of Oct-4, SSEA-4 and TRal-60, for example at the polypeptide level. They may display transcript expression of one or both of OCT4 and SOX2, but at reduced levels compared to embryonic stem cells such as hES3 human ESCs. The levels of expression may be 2 times lower, 5 times lower or 10 times lower or more compared to embryonic stem cells.

The obtained mesenchymal stem cells may display a “typical” MSC-like surface antigen profile. They may for example show expression of one or more markers associated with mesenchymal stem cells. These may include expression of any one or more of the following: CD29, CD44, CD49a, CD49e, CD105, CD166, MHC I. The pre-natal mesenchymal stem cells may for example show reduced or absent expression of any one or more markers whose absence of expression is associated with mesenchymal stem cells. Thus, the pre-natal mesenchymal stem cells may display reduced or lack of expression of any one or more of HLA-DR, CD34 and CD45. The pre-natal mesenchymal stem cells may in particular comprise CD29+, CD44+, CD49a+ CD49e+, CD105+, CD166+, MHC I+, CD34⁻ and CD45⁻ cells.

The mesenchymal stem cell may be negative for mouse-specific c-mos repeat sequences and positive for human specific alu repeat sequences. It may be capable of undergoing any one or more of osteogenesis, adipogenesis or chondrogenesis, and in particular it may be capable of differentiating into any one or more of osteocytes, adipocytes or chondrocytes.

Differentiation Potential

The mesenchymal stem cells obtained may be differentiated into any mesenchymal lineage, using methods known in the art and described below. Thus, the mesenchymal stem cells obtained by the methods and compositions described here may display a differentiation potential that include adipogenesis, chondrogenesis and osteogenesis⁹.

Proliferative Capacity

The mesenchymal stem cells obtained as described, e.g., hESC-MSCs, can have a substantial proliferative capacity in vitro. In some embodiments, the mesenchymal stem cells obtained may undergo at least 10 population doublings while maintaining a normal diploid karyotype. The mesenchymal stem cells may be capable of undergoing at least 20-30 population doublings while maintaining a normal diploid karyotype. In some embodiments, the mesenchymal stem cells display a stable gene expression and surface antigen profile throughout this time.

The mesenchymal stem cells obtained may be such that they do not display any defects, such as chromosomal aberrations and/or alterations in gene expression. In some embodiments, such defects are not evident until after 10 passages, such as after 13 passages, for example after 15 passages.

Homogeneity

The mesenchymal stem cells obtained may display a high degree of uniformity. In other words, the mesenchymal stem cells obtained from different pre-natal sources may display one or more, such as a plurality, of uniform or distinct characteristics that are shared with each other. They may display one or more, such as a plurality, of uniform or distinct characteristics that are shared with other mesenchymal stem cells, such as a human embryonic stem cell derived mesenchymal stem cells (hESC-MSCs), as for example described in WO2007/027157 or WO2007/027156.

For example, the mesenchymal stem cells may be such that any two or more, such as all, mesenchymal stem cells selected by the method exhibit substantially identical gene expression profiles. The gene expression correlation coefficient between any two or more isolates of mesenchymal stem cells obtained by the method may be greater than 0.8, such as greater than 0.85 or 0.9. The gene expression correlation coefficient between any two or more different passages, such as successive passages, of mesenchymal stem cells obtained by the method may be greater than 0.8, such as greater than 0.85 or 0.9. The gene expression correlation coefficient between a mesenchymal stem cell obtained by the method and hESC-MSCs, such as for example described in WO2007/027157 or WO2007/027156, may be greater than 0.8, such as greater than 0.85 or 0.9.

Any two or more, such as all, isolates of mesenchymal stem cells obtained by the method may be substantially similar or identical (such as homogenous) with each other.

This is described in more detail in the section below.

Telomerase Activity

The mesenchymal stem cells so derived may comprise telomerase activity. The telomerase activity may be elevated or up-regulated compared to a control cell such as a cell which is not a mesenchymal stem cell. For example, the control cell may comprise a differentiated cell, such as a differentiated cell in the mesenchymal lineage, for example, an osteocyte, adipocyte or chondrocyte.

Telomerase activity may be determined by means known in the art, for example, using TRAP activity assay (Kim et al., Science 266:2011, 1997), using a commercially available kit (TRAPeze® XK Telomerase Detection Kit, Cat. s7707; Intergen Co., Purchase N.Y.; or TeloTAGGG™ Telomerase PCR ELISA plus, Cat. 2,013,89; Roche Diagnostics, Indianapolis). hTERT expression can also be evaluated at the mRNA level by RT-PCR. The LightCycler TeloTAGGG™ hTERT quantification kit (Cat. 3,012,344; Roche Diagnostics) is available commercially for research purposes.

For example, relative telomerase activity may be measured by real time quantitative telomeric repeat amplification protocol. This qPCR-based assay quantifies product generated in vitro by telomerase activity present in the samples. The relative telomerase activity which is directly proportional to the amount of telomerase products may be assessed by the threshold cycle number (or Ct value) for one μg protein cell lysate. Hues9.E1 refers to a previously described hESC-MSCs line and HEK is a human embryonic kidney cell line. The Ct value for each fetal MSCs may be taken as a mean for multiple passages, for example, 2, 3, 4 etc passages. The passages may for example comprise three passages, such as P16, P18, and P20. For the control cells, such as Hues9.E1 the mean may be for two passages, such as P20 and P22. The assay may be performed multiple times, such as in triplicate, for each passage.

A specific example of a telomerase detection method is provided in the Examples.

The mesenchymal stem cells derived by the methods described here may have a Ct value, as assayed by such a method, of 25 or more, such as 26 or more or 27 or more. The Ct value may be for example 28 or more, 29 or more, 30 or more, 31 or more or 32 or more.

Cardioprotective Ability

The mesenchymal stem cell so derived or a medium conditioned by such a cell may comprise cardioprotective ability, as described in the Examples. The cardioprotection may comprise restoration or maintenance of cardiac function during ischemia and/or reperfusion.

Cardioprotection may be assayed in any suitable model system, such as a mouse model of acute myocardial infarction (AMI). In such an assay, AMI is induced in mice by permanent ligation of the left anterior descending coronary artery as described in Salto-Tellez M, Yung Lim S, El-Oakley R M, Tang T P, ZA A L, et al. (2004) Myocardial infarction in the C57BL/6J mouse: a quantifiable and highly reproducible experimental model. Cardiovasc Pathol 13: 91-97.

100 μl of 10× concentrated conditioned medium or non-conditioned medium (control) made as described above is then administered to the mice via an osmotic pump placed at the jugular vein over the next 72 hours. Heart function in these mice is assessed by MRI three weeks later.

Cardioprotection may also be assayed in a pig/mouse model of myocardial ischemia/reperfusion (MI/R) injury (Timmers L, Lim S-K, Arslan F, et al. Reduction of myocardial infarct size by human mesenchymal stem cell conditioned medium. Stem Cell Research. 2008; 1:129-137). In this assay, injury is induced by a temporary occlusion of the left circumflex artery in pig or the LAD in mouse followed by removal of occlusion to initiate reperfusion.

The cardioprotection assays described above may also be used to test for cardioprotection especially in chronic ischemia. This and the other MI/R injury model essentially evaluate cardioprotection in different clinical indications.

The mesenchymal stem cell so derived or a medium conditioned by such a cell may be capable of alleviating reperfusion injury. This may be assayed using a porcine model of ischemia-reperfusion as described in WO2008/020815.

A brief protocol for assaying cardioprotective ability of pre-natal mesenchymal stem cell conditioned medium follows. MI may be induced by 30 minutes occlusion of left coronary artery (LCA) by ligating the artery with a suture. Subsequent reperfusion may be initiated by releasing the suture. Five minutes before reperfusion, mice may be intravenously infused with 200 μl saline diluted conditioned medium containing 3 μg protein for Hues9.E1 (hESC-MSC) CM or 150 μg protein for fetal MSC CM via the tail vein. Control animals may be infused with 200 μl saline. After 24 hours reperfusion, infarct size (1S) as a percentage of the area at risk (AAR) may be assessed using Evans' blue dye injection and TTC staining as described previously in reference 26.

Briefly, just before excision of the heart for analysis, the LCA may be re-ligated as in the induction of ischemia, Evans blue dye may be infused into the aorta and the AAR may be defined by the area not stained by Evans' blue dye. The heart may then be excised and cross sections of the heart may be stained with TTC. Viable myocardium is stained red by TTC while non-viable myocardium is not stained. Relative infarct size may be measured as the area of non-viable myocardium not stained by TTC relative to the AAR risk defined by the area not stained by Evans' blue dye.

Infarct Size

The mesenchymal stem cell so derived or a medium conditioned by such a cell may have the ability to reduce infarct size. The infarct size may be reduced by 10% or more, 20% or more, 30% or more, 40% or more, 50% or more, 60% or more or 70% or more compared with an animal that is treated with a non-conditioned medium or saline.

Assay for Infarct Size

Infarct size may for example be assayed using the following method.

Just prior to excision of the heart, the LCxCA (pigs) or LCA (mice) is religated at exactly the same spot as for the induction of the MI. Evans blue dye is infused through the coronary system to delineate the area at risk (AAR).

The heart is then excised, the LV is isolated and cut into 5 slices from apex to base. The slices are incubated in 1% triphenyltetrazolium chloride (TTC, Sigma-Aldrich Chemicals, Zwijndrecht, the Netherlands) in 37° C. Sørensen buffer (13.6 g/L KH₂PO₄+17.8 g/L Na₂H PO₄.2H₂O, pH 7.4) for 15 minutes to discriminate infarct tissue from viable myocardium.

All slices are scanned from both sides, and in each slide, the infarct area is compared with area at risk and the total area by use of digital planimetry software (Image J). After correction for the weight of the slices, infarct size is calculated as a percentage of the AAR and of the LV.

Oxidative Stress

The mesenchymal stem cells produced by the method described here may be capable of reducing oxidative stress.

The oxidative stress may be reduced by 10% or more, 20% or more, 25% or more, 30% or more, 40% or more, 50% or more, 60% or more or 70% or more compared with an animal that is treated with a non-conditioned medium or saline.

The reduction of oxidative stress may be assayed in an in vitro assay of hydrogen peroxide (H₂O₂)-induced cell death.

Assay for Oxidative Stress

The reduction of oxidative stress may for example be assayed using an in vitro assay of hydrogen peroxide (H₂O₂)-induced cell death. In summary, hydrogen peroxide (H₂O₂)-mediated oxidative stress is induced in human leukemic CEM cells and cell viability is monitored by Trypan blue-exclusion. Human leukemic CEM cells are incubated with conditioned medium or mesenchymal stem cell (with saline as a control) and treated with 50 μM H₂O₂ to induce oxidative stress. Cell viability is assessed using Trypan Blue exclusion at 12, 24, 36 and 48 hours after H₂O₂ treatment.

The reduction of oxidative stress may further be assayed using an in vivo assay of DNA oxidation. In vivo oxidative stress may also be assayed as follows. Pigs are treated with the particle, conditioned medium or mesenchymal stem cell (with saline as a control). Tissue sections of pig heart are obtained. Nuclear oxidative stress in tissue sections of treated and untreated pigs is quantified by 8-OHdG immunostaining for oxidized DNA. The tissue sections are assayed for intense nuclear staining indicative of DNA oxidation and oxidative stress.

Homogeneity

The mesenchymal stem cells produced by the method described here may be similar or identical (such as homogenous) in nature. That is to say, mesenchymal stem cell (MSC) clones isolated by the protocol may show a high degree of similarity or identity with each other, whether phenotypically or otherwise.

Similarity or identity may be gauged by a number of ways and measured by one or more characteristics. For example, the clones may be similar or identical in gene expression. The method may be such that any two or more mesenchymal stem cells selected by the method exhibit substantially identical or similar gene expression profiles, that is to say, a combination of the identity of genes expressed and the level to which they are expressed. For example, substantially all of the mesenchymal stem cells isolated may exhibit substantially identical or similar gene expression profiles.

Homogeneity of gene expression may be measured by a number of methods. Genome-wide gene profiling may be conducted using, for example, array hybridisation of extracted RNA as described in the Examples. Total RNA may be extracted and converted into cDNA, which is hybridised to an array chip comprising a plurality of gene sequences from a relevant genome. The array may comprise NCBI Reference Sequence (RefSeq) genes, which are well characterised genes validated, annotated and curated on an ongoing basis by National Center for Biotechnology Information (NCBI) staff and collaborators.

Gene expression between samples may then be compared using analysis software. In one embodiment, the similarity or identity of gene expression may be expressed as a “correlation coefficient”. In such measures, a high correlation coefficient between two samples indicates a high degree of similarity between the pattern of gene expression in the two samples. Conversely, a low correlation coefficient between two samples indicates a low degree of similarity between the pattern of gene expression in the two samples. Normalisation may be conducted to remove systematic variations or bias (including intensity bias, spatial bias, plate bias and background bias) prior to data analysis.

Correlation tests are known in the art and include a T-test and Pearson's test, as described in for example Hill, T. & Lewicki, P. (2006). Statistics: Methods and Applications. StatSoft, Tulsa, Okla., ISBN: 1884233597 (also StatSoft, Inc. (2006). Electronic Statistics Textbook. Tulsa, Okla.: StatSoft. WEB: http://www.statsoft.com/textbook/stathome.html). Reference is made to Khojasteh et al., 2005, A stepwise framework for the normalization of array CGH data, BMC Bioinformatics 2005, 6:274. An Intra-class correlation coefficient (ICC) may also be conducted, as described in Khojasteh et al, supra.

For example, a Pearson's test may be conducted to generate a Pearson's correlation coefficient. A correlation coefficient of 1.0 indicates an identical gene expression pattern.

For such purposes, the cDNA may be hybridised to a Sentrix HumanRef-8 Expression BeadChip and scanned using a Illumina BeadStation 500×. The data may be extracted, normalised and analysed using Illumina BeadStudio (Illumina, Inc, San Diego, Calif., USA). It will be clear to the reader however that any suitable chip and scanning hardware and software (which outputs a correlation measurement) may be used to assay similarity of gene expression profile.

The gene expression correlation coefficient between any two isolates as for example measured by the above means may be 0.65 or more. The gene expression correlation coefficient may be 0.70 or more, such as 0.80 or more, such as 0.85 or more, such as 0.90 or more. The coefficient may be 0.91 or more, 0.92 or more, 0.93 or more, 0.94 or more, 0.95 or more, 0.96 or more, 0.97 or more, 0.98 or more, 0.99 or more or 1.0.

In some embodiments, the method described here generates mesenchymal stem cells whose gene expression correlation coefficient between any two or more isolates of mesenchymal stem cells so obtained is in the same order as, or slightly less than, the correlation coefficient between technical replicates of the same RNA sample, performed a period of time apart such as 1 month apart. For example, the gene expression correlation coefficient between any two or more isolates of mesenchymal stem cells may be 0.70 or more, such as 0.80 or more, such as 0.85 or more, such as 0.90 or more. The coefficient may be 0.91 or more, 0.92 or more, 0.93 or more, 0.94 or more, 0.95 or more, 0.96 or more, 0.97 or more, 0.98 or more, 0.99 or more or 1.0.

The gene expression correlation coefficients may be in such ranges for cells which have undergone the derivation, selection or sorting procedure described above. The gene expression correlation coefficient between the majority of isolates, such as all isolates, may be in such ranges.

Thus, as shown in the Examples, the correlation coefficient shows a high degree of similarity between the five mesenchymal stem cell cultures obtained. The correlation value of the gene expression profile between different passages of each culture is greater than 0.9. The correlation value of the gene expression profile between the five cultures is greater than 0.9.

Accordingly, we provide for a method of generating mesenchymal stem cells which are substantially similar or identical (such as homogenous) with each other. The isolates may display a near identical gene expression profile.

As well as the “internal” homogeneity described above (i.e., homogeneity between the isolates of mesenchymal stem cells from the method), homogeneity may also be assessed between such isolates and other cells or cell types. In particular, comparisons may be made with mesenchymal stem cells derived by other methods, such as a human ESC-MSC culture (for example as described in WO2007/027157 or WO2007/027156). In some embodiments, therefore, the mesenchymal stem cells obtained by the methods and compositions described here display a gene expression profile which is similar to, homogenous with, or identical with such a human ESC-MSC culture. Thus, the mesenchymal stem cells obtained may show a correlation coefficient of gene expression of greater than 0.5, such as greater than 0.6, such as greater than 0.7, such as greater than 0.8, such as greater than 0.9, as with such a human ESC-MSC culture (for example as described in WO2007/027157 or WO2007/027156).

Thus, as shown in the Examples, pairwise comparision of gene expression between five independently derived pre-natal mesenchymal stem cell populations and human ESC-MSC culture samples are found to be similar with a correlation coefficient of greater than 0.9.

### Pre-Natal Mesenchymal Stem Cell Cultures and Cell Lines

It will be evident that such a mesenchymal stem cell that is obtained by the methods described here may be maintained as a cell or developed into a cell culture or a cell line.

Accordingly, in this document, and where appropriate, the term “mesenchymal stem cell” should be taken also to include reference to a corresponding cell culture, i.e., a mesenchymal stem cell culture or a corresponding cell line, i.e., a mesenchymal stem cell line. In general, the mesenchymal stem cell, cell culture or cell line may be maintained in culture in the same or similar conditions and culture media as described above for derivation.

### Pre-Natal Mesenchymal Stem Cell Conditioned Medium

We further provide a medium which is conditioned by culture of the pre-natal mesenchymal stem cells.

Such a conditioned medium may comprise molecules secreted, excreted, released, or otherwise produced by the pre-natal mesenchymal stem cells. The conditioned medium may comprise one or more molecules of the mesenchymal stem cells, for example, polypeptides, nucleic acids, carbohydrates or other complex or simple molecules. The conditioned medium may also comprise one or more activities of the mesenchymal stem cells. The conditioned medium may be used in place of, in addition to, or to supplement the mesenchymal stem cells themselves. Thus, any purpose for which mesenchymal stem cells are suitable for use in, conditioned media may similarly be used for that purpose.

Such a conditioned medium, and combinations of any of the molecules comprised therein, including in particular proteins or polypeptides, may be used to supplement the activity of, or in place of, the pre-natal mesenchymal stem cells, for the purpose of for example treating or preventing a disease. Thus, where it is stated that the mesenchymal stem cells obtained by the methods described here may be used for a particular purpose, it will be evident that media conditioned by such mesenchymal stem cells may be equally used for that purpose.

Conditioned medium may be made by any suitable method. For example, it may be made by culturing mesenchymal stem cells in a medium, such as a cell culture medium, for a predetermined length of time. Any number of methods of preparing conditioned medium may be employed, including the “Conditioned Medium Preparation Protocol” set out below.

The mesenchymal stem cells may in particular comprise those produced by any of the methods described in this document. The conditioned medium will comprise polypeptides secreted by the mesenchymal stem cells, as described in the Examples.

A particular example of a protocol for producing conditioned medium, which is not intended to be limiting, is as follows.

An example protocol for preparing conditioned medium from pre-natal mesenchymal stem cells may comprise a “Conditioned Medium Preparation Protocol”, which is as follows:

Pre-Natal Mesenchymal Stem Cell Conditioned Medium Preparation Protocol

The secretion may be prepared by growing the pre-natal, such as foetal, MSCs in a chemically defined serum free culture medium for three days as described above and in the Examples, and also in reference 25.80% confluent cultures may be washed three times with PBS, and then cultured overnight in a chemically defined medium consisting of DMEM media without phenol red supplemented with 1× Insulin-Transferrin-Selenoprotein, 10 ng/ml Recombinant Human FGF2, 10 ng/ml Recombinant Human EGF, 1× glutamine-penicillin-streptomycin, and 55 μM β-mercaptoethanol overnight. The cultures may then be washed three times with PBS and fresh chemically defined medium may then be added. All medium components may be obtained from Invitrogen. The cultures may be maintained in this medium for three days. This conditioned medium (CM) may then be collected, clarified by centrifugation, concentrated 50 times using tangential flow filtration with MW cut-off of 100 kDa (Satorius, Goettingen, Germany) and sterilized by filtration through a 220 nm filter.

The conditioned medium may be used in therapy as is, or after one or more treatment steps. For example, the conditioned medium may be UV treated, filter sterilised, etc. One or more purification steps may be employed. In particular, the conditioned media may be concentrated, for example by dialysis or ultrafiltration. For example, the medium may be concentrated using membrane ultrafiltration with a nominal molecular weight limit (NMWL) of for example 3K.

For the purposes described in this document, for example, treatment or prevention of disease, a dosage of conditioned medium comprising 15-750 mg protein/100 kg body weight may be administered to a patient in need of such treatment.

### Pre-Natal Mesenchymal Stem Cell Particle

We describe a particle which is derivable from a pre-natal mesenchymal stem cell (MSC). Such a particle is referred to in this document as a “pre-natal mesenchymal stem cell particle”.

The pre-natal mesenchymal stem cell particle may be derivable from the pre-natal MSC by any of several means, for example by secretion, budding or dispersal from the pre-natal MSC. For example, the pre-natal mesenchymal stem cell particle may be produced, exuded, emitted or shed from the pre-natal MSC. Where the pre-natal MSC is in cell culture, the pre-natal mesenchymal stem cell particle may be secreted into the cell culture medium.

The pre-natal mesenchymal stem cell particle may in particular comprise a vesicle. The pre-natal mesenchymal stem cell particle may comprise an exosome. The pre-natal mesenchymal stem cell particle described here may comprise any one or more of the properties of the exosomes described herein.

The pre-natal mesenchymal stem cell particle may comprise vesicles or a flattened sphere limited by a lipid bilayer. The pre-natal mesenchymal stem cell particle may comprise diameters of 40-100 nm. The pre-natal mesenchymal stem cell particle may be formed by inward budding of the endosomal membrane. The pre-natal mesenchymal stem cell particle may have a density of ˜1.13-1.19 g/ml and may float on sucrose gradients. The pre-natal mesenchymal stem cell particle may be enriched in cholesterol and sphingomyelin, and lipid raft markers such as GM1, GM3, flotillin and the src protein kinase Lyn. The pre-natal mesenchymal stem cell particle may comprise one or more proteins present in pre-natal mesenchymal stem cells or pre-natal mesenchymal stem cell conditioned medium (MSC-CM), such as a protein characteristic or specific to the pre-natal MSC or pre-natal MSC-CM. They may comprise RNA, for example miRNA.

We provide a pre-natal mesenchymal stem cell particle which comprises one or more genes or gene products found in pre-natal MSCs or medium which is conditioned by culture of pre-natal MSCs. The pre-natal mesenchymal stem cell particle may comprise molecules secreted by the pre-natal MSC. Such a pre-natal mesenchymal stem cell particle, and combinations of any of the molecules comprised therein, including in particular proteins or polypeptides, may be used to supplement the activity of, or in place of, the pre-natal MSCs or medium conditioned by the pre-natal MSCs for the purpose of for example treating or preventing a disease.

The pre-natal mesenchymal stem cell particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the pre-natal mesenchymal stem cell particle may comprise one or more tetraspanins. The pre-natal mesenchymal stem cell particles may comprise mRNA and/or microRNA.

The term “particle” as used in this document may be taken to mean a discrete entity. The particle may be something that is isolatable from a pre-natal mesenchymal stem cell (pre-natal MSC) or pre-natal mesenchymal stem cell conditioned medium (pre-natal MSC-CM). The pre-natal mesenchymal stem cell particle may be responsible for at least an activity of the pre-natal MSC or pre-natal MSC-CM. The pre-natal mesenchymal stem cell particle may be responsible for, and carry out, substantially most or all of the functions of the pre-natal MSC or pre-natal MSC-CM. For example, the pre-natal mesenchymal stem cell particle may be a substitute (or biological substitute) for the pre-natal MSC or pre-natal MSC-CM.

The pre-natal mesenchymal stem cell particle may be used for any of the therapeutic purposes that the pre-natal MSC or pre-natal MSC-CM may be put to use.

The pre-natal mesenchymal stem cell particle preferably has at least one property of a pre-natal mesenchymal stem cell. The pre-natal mesenchymal stem cell particle may have a biological property, such as a biological activity. The pre-natal mesenchymal stem cell particle may have any of the biological activities of an pre-natal MSC. The pre-natal mesenchymal stem cell particle may for example have a therapeutic or restorative activity of an pre-natal MSC.

The Examples show that media conditioned by pre-natal MSCs (such as pre-natal mesenchymal stem cell conditioned media or pre-natal MSC-CM, as described below) comprise biological activities of pre-natal MSC and are capable of substituting for the pre-natal MSCs themselves. The biological property or biological activity of an pre-natal MSC may therefore correspond to a biological property or activity of a pre-natal mesenchymal stem cell conditioned medium. Accordingly, the pre-natal mesenchymal stem cell particle may comprise one or more biological properties or activities of a pre-natal mesenchymal stem cell conditioned medium (pre-natal MSC-CM).

The conditioned cell culture medium such as a Pre-Natal Mesenchymal Stem Cell Conditioned Medium (pre-Natal MSC-CM) may be obtained by culturing a pre-natal mesenchymal stem cell (pre-natal MSC), a descendent thereof or a cell line derived therefrom in a cell culture medium; and isolating the cell culture medium. The pre-natal mesenchymal stem cell may be produced by a process comprising obtaining a cell by dispersing a embryonic stem (ES) cell colony. The cell, or a descendent thereof, may be propagated in the absence of co-culture in a serum free medium comprising FGF2. Further details are provided elsewhere in this document.

Isolation of Pre-Natal Mesenchymal Stem Cell Particle

The pre-natal MSC particle may be produced or isolated in a number of ways. Such a method may comprise isolating the particle from a pre-natal mesenchymal stem cell (pre-natal MSC). Such a method may comprise isolating the pre-natal MSC particle from a pre-natal mesenchymal stem cell conditioned medium (pre-natal MSC-CM).

The pre-natal MSC particle may be isolated for example by being separated from non-associated components based on any property of the pre-natal MSC particle. For example, the pre-natal MSC particle may be isolated based on molecular weight, size, shape, composition or biological activity.

The conditioned medium may be filtered or concentrated or both during, prior to or subsequent to separation. For example, it may be filtered through a membrane, for example one with a size or molecular weight cut-off. It may be subject to tangential force filtration or ultrafiltration.

For example, filtration with a membrane of a suitable molecular weight or size cutoff, as described in the Assays for Molecular Weight elsewhere in this document, may be used.

The conditioned medium, optionally filtered or concentrated or both, may be subject to further separation means, such as column chromatography. For example, high performance liquid chromatography (HPLC) with various columns may be used. The columns may be size exclusion columns or binding columns.

One or more properties or biological activities of the pre-natal MSC particle may be used to track its activity during fractionation of the pre-natal mesenchymal stem cell conditioned medium (pre-natal MSC-CM). As an example, light scattering, refractive index, dynamic light scattering or UV-visible detectors may be used to follow the pre-natal MSC particles. For example, a therapeutic activity such as cardioprotective activity may be used to track the activity during fractionation.

The following paragraphs provide a specific example of how a pre-natal MSC mesenchymal stem cell particle such as an exosome may be obtained.

A pre-natal mesenchymal stem cell particle may be produced by culturing mesenchymal stem cells in a medium to condition it. The pre-natal mesenchymal stem cells may comprise HuES9.E1 cells. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof.

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more.

The conditioned medium may be subject to liquid chromatography such as HPLC. The conditioned medium may be separated by size exclusion. Any size exclusion matrix such as Sepharose may be used. As an example, a TSK Guard column SWXL, 6×40 mm or a TSK gel G4000 SWXL, 7.8×300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector.

Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The rₕ of particles in this peak is about 55-65 nm. Such fractions comprise pre-natal MSC mesenchymal stem cell particles such as exosomes.

### Delivery of Pre-Natal MSC Particles

The pre-natal MSC particles as described in this document may be delivered to the human or animal body by any suitable means.

We therefore describe a delivery system for delivering a pre-natal MSC particle as described in this document to a target cell, tissue, organ, animal body or human body, and methods for using the delivery system to deliver pre-natal MSC particles to a target.

The delivery system may comprise a source of pre-natal MSC particles, such as a container containing the pre-natal MSC particles. The delivery system may comprise a dispenser for dispensing the pre-natal MSC particles to a target.

Accordingly, we provide a delivery system for delivering a pre-natal MSC particle, comprising a source of pre-natal MSC particles as described in this document together with a dispenser operable to deliver the pre-natal MSC particles to a target.

We further provide for the use of such a delivery system in a method of delivering pre-natal MSC particles to a target.

Delivery systems for delivering fluid into the body are known in the art, and include injection, surgical drips, cathethers (including perfusion cathethers) such as those described in U.S. Pat. No. 6,139,524, for example, drug delivery catheters such as those described in U.S. Pat. No. 7,122,019.

Delivery to the lungs or nasal passages, including intranasal delivery, may be achieved using for example a nasal spray, puffer, inhaler, etc as known in the art (for example as shown in U.S. Design Pat. D544,957.

Delivery to the kidneys may be achieved using an intra-aortic renal delivery catheter, such as that described in U.S. Pat. No. 7,241,273.

It will be evident that the particular delivery should be configurable to deliver the required amount of pre-natal MSC particles at the appropriate interval, in order to achieve optimal treatment.

The pre-natal MSC particles may for example be used for the treatment or prevention of atherosclerosis. Here, perfusion of pre-natal MSC particles may be done intravenously to stabilize atherosclerotic plaques or reduce inflammation in the plaques. The pre-natal MSC particles may be used for the treatment or prevention of septic shock by intravenous perfusion.

The pre-natal MSC particles may be used for the treatment or prevention of heart failure. This may be achieved by chronic intracoronary or intramyocardially perfusion of pre-natal MSC particles to retard remodeling or retard heart failure. The pre-natal MSC particles may be used for the treatment or prevention of lung inflammation by intranasal delivery.

The pre-natal MSC particles may be used for the treatment or prevention of dermatological conditions e.g. psoriasis. Long term delivery of pre-natal MSC particles may be employed using transdermal microinjection needles until the condition is resolved.

It will be evident that the delivery method will depend on the particular organ to which the pre-natal MSC particles is to be delivered, and the skilled person will be able to determine which means to employ accordingly.

As an example, in the treatment of cardiac inflammation, the pre-natal MSC particles may be delivered for example to the cardiac tissue (i.e., myocardium, pericardium, or endocardium) by direct intracoronary injection through the chest wall or using standard percutaneous catheter based methods under fluoroscopic guidance for direct injection into tissue such as the myocardium or infusion of an inhibitor from a stent or catheter which is inserted into a bodily lumen.

Any variety of coronary catheter, or a perfusion catheter, may be used to administer the pre-natal MSC particles. Alternatively the pre-natal MSC particles may be coated or impregnated on a stent that is placed in a coronary vessel.

### Maintenance as Cell Culture

The mesenchymal stem cells may be plated and maintained as a cell culture.

The cells may be plated onto a culture vessel or substrate such as a gelatinized plate. The cells may be grown and propagated without the presence of co-culture, e.g., in the absence of feeder cells.

The cells in the cell culture may be grown in a serum-free medium. The medium may be supplemented by one or more growth factors such as epidermal growth factor (EGF), fibroblast growth factor 2 (FGF2) and optionally platelet-derived growth factor AB (PDGF AB), at for example 5 ng/ml, 10 ng/ml, 15 ng/ml, 20 ng/ml, 25 ng/ml or 30 ng/ml. The cells in the cell culture may be split or subcultured when confluent.

They may be split by any suitable means, such as treatment with trypsin, washing and replating. The split ratio may comprise for example 1:4.

The mesenchymal stem cells may be maintained as a cell line.

### Absence of Co-Culture

In some embodiments, our methods involve culturing cells in the absence of co-culture.

The term “co-culture” refers to a mixture of two or more different kinds of cells that may be grown together, for example, stromal feeder cells.

Thus, in typical ES cell culture, the inner surface of the culture dish is usually coated with a feeder layer of mouse embryonic skin cells that have been treated so they will not divide. The feeder layer provides an adherent surface to enable the ES cells to attach and grow. In addition, the feeder cells release nutrients into the culture medium which may be required for ES cell growth.

In the methods and compositions described here, the mesenchymal stem cells may be cultured in the absence of such co-culture. Thus, the cells may be cultured as a monolayer or in the absence of feeder cells.

The mesenchymal stem cells may be cultured on a culture substrate. The culture substrate may comprise a tissue culture vessel, such as a Petri dish. The vessel may be pre-treated. The cells may be plated onto, and grow on, a gelatinised tissue culture plate.

An example protocol for the gelatin coating of dishes follows. A solution of 0.1% gelatin in distilled water is made and autoclaved. This may be stored at room temp. The bottom of a tissue culture dish is covered with the gelatin solution and incubated for 5-15 min. Remove gelatin and plates are ready to use. Medium should be added before adding cells to prevent hypotonic lysis.

### Serum Free Media

The mesenchymal stem cells may be cultured in a medium which may comprise a serum-free medium.

The term “serum-free media” may comprise cell culture media which is free of serum proteins, e.g., fetal calf serum. Serum-free media are known in the art, and are described for example in U.S. Pat. Nos. 5,631,159 and 5,661,034. Serum-free media are commercially available from, for example, Gibco-BRL (Invitrogen).

The serum-free media may be protein free, in that it may lack proteins, hydrolysates, and components of unknown composition. The serum-free media may comprise chemically-defined media in which all components have a known chemical structure. Chemically-defined serum-free media may be used as it provides a completely defined system which eliminates variability allows for improved reproducibility and more consistent performance, and decreases possibility of contamination by adventitious agents.

The serum-free media may comprise Knockout DMEM media (Invitrogen-Gibco, Grand Island, N.Y.).

The serum-free media may be supplemented with one or more components, such as serum replacement media, at a concentration of for example, 5%, 10%, 15%, etc. The serum-free media may for example be supplemented with 10% serum replacement media from Invitrogen-Gibco (Grand Island, N.Y.).

### Growth Factor

The serum-free medium in mesenchymal stem cells are cultured may comprise one or more growth factors. A number of growth factors are known in the art, including PDGF, EGF, TGF-a, FGF, NGF, Erythropoietin, TGF-b, IGF-I and IGF-II.

The growth factor may comprise fibroblast growth factor 2 (FGF2). The medium may also contain other growth factors such as epidermal growth factor (EGF) or platelet-derived growth factor AB (PDGF AB). Both of these growth factors are known in the art. The method may comprise culturing cells in a medium comprising both FGF2 and EGF.

FGF2 is a wide-spectrum mitogenic, angiogenic, and neurotrophic factor that is expressed at low levels in many tissues and cell types and reaches high concentrations in brain and pituitary. FGF2 has been implicated in a multitude of physiologic and pathologic processes, including limb development, angiogenesis, wound healing, and tumor growth. FGF2 may be obtained commercially, for example from Invitrogen-Gibco (Grand Island, N.Y.).

Platelet Derived Growth Factor (PDGF) is a potent mitogen for a wide range of cell types including fibroblasts, smooth muscle and connective tissue. PDGF, which is composed of a dimer of two chains termed the A chain and B chain, can be present as AA or BB homodimers or as an AB heterodimer. Human PDGF-AB is a 25.5 kDa homodimer protein consisting of 13.3 kDa A chain and 12.2 B chain. PDGF AB may be obtained commercially, for example from Peprotech (Rocky Hill, N.J.).

Epidermal growth factor or EGF is a growth factor that plays an important role in the regulation of cell growth, proliferation, and differentiation by binding to its receptor EGFR. Human EGF is a 6045-Da protein with 53 amino acid residues and three intramolecular disulfide bonds. EGF acts by binding with high affinity to epidermal growth factor receptor (EGFR) on the cell surface and stimulating the intrinsic protein-tyrosine kinase activity of the receptor. The tyrosine kinase activity, in turn, initiates a signal transduction cascade that results in a variety of biochemical changes within the cell—a rise in intracellular calcium 120 levels, increased glycolysis and protein synthesis, and increases in the expression of certain genes including the gene for EGFR—that ultimately lead to DNA synthesis and cell proliferation. EGF may be obtained commercially from a number of manufacturers.

The growth factor(s), such as EGF, FGF2 and optionally PDGF AB, may be present in the medium at concentrations of about 100 pg/ml, about 500 pg/ml, about 1 ng/ml, about 2 ng/ml, about 3 ng/ml, about 4 ng/ml or about 5 ng/ml. In some embodiments, the medium contains FGF2 at about 20 ng/ml. The medium may also contain EGF, such as at about 20 ng/ml.

### Splitting Cells

Cells in culture will generally continue growing until confluence, when contact inhibition causes cessation of cell division and growth. Such cells may then be dissociated from the substrate or flask, and “split”, subcultured or passaged, by dilution into tissue culture medium and replating.

The methods and compositions described here may therefore comprise passaging, or splitting during culture. The cells in the cell culture may be split at a ratio of 1:2 or more, such as 1:3, 1:4, 1:5 or more. The term “passage” designates the process consisting in taking an aliquot of a confluent culture of a cell line, in inoculating into fresh medium, and in culturing the line until confluence or saturation is obtained.

### Selection, Screening or Sorting Step

In some embodiments, the derivation method may further comprise a selection or sorting step, to further isolate or select for mesenchymal stem cells.

The selection or sorting step may comprise selecting mesenchymal stem cells (MSC) from the originating cell culture by means of one or more surface antigen markers. The use of a selection or sorting step may further enhance the stringency of sorting and selection specificity for mesenchymal stem cells and may furthermore potentially reduce possible contamination from the starting material. This may then further reduce the risk of teratoma formation and further increase the clinical relevance of the protocol we describe.

A number of methods are known for selection or sorting based on antigen expression, and any of these may be used in the selection or sorting step described here. For example, the selection or sorting may be achieved by means of fluorescence activated cell sorting (FACS). Thus, as known in the art, FACS involves exposing cells to a reporter, such as a labelled antibody, which binds to and labels antigens expressed by the cell. Methods of production of antibodies and labelling thereof to form reporters are known in the art, and described for example in Harlow and Lane. The cells are then passed through a FACS machine, which sorts the cells from each other based on the labelling.

The sorting may be based on any number of surface antigens known or suspected to be associated with mesenchymal stem cells.

The selection or sorting step may positively select for mesenchymal stem cells based on the expression of antigens which are identified as expressed on mesenchymal stem cells.

Alternatively, or in addition, the selection or sorting step may negatively select against antigens based on surface antigens that are not mesenchymal stem cells.

For example, CD24 may be used as a negative selection or sorting marker either on its own, or in conjunction with CD105 as a positive selectable marker for isolating mesenchymal stem cells.

### Maintenance of Self-Renewal

The mesenchymal stem cells may be able to maintain self-renewal without the requirement for transformation.

Thus, for example, known transformation treatments such as fusion with immortalised cells such as tumour cells or tumour cell lines, viral infection of a cell line with tranforming viruses such as SV40, EBV, HBV or HTLV-1, transfection with specially adapted vectors, such as the SV40 vector comprising a sequence of the large T antigen (R. D. Berry et al., Br. J. Cancer, 57, 287-289, 1988), telomerase (Bodnar-A-G. et. al., Science (1998) 279: p. 349-52) or a vector comprising DNA sequences of the human papillomavirus (U.S. Pat. No. 5,376,542), introduction of a dominant oncogene, or by mutation are therefore not required in the methods described here for making mesenchymal stem cells.

The mesenchymal stem cells and cell lines (or the differentiated cells derived from them) may be such that they do not display one or more characteristics of embryonic stem cells. Such characteristics may include expression of the OCT4 gene, the SSEA-4 gene, the Tra1-60 gene and alkaline phosphatase activity. The mesenchymal stem cell may exhibit reduced expression of one or more characteristic markers of pluripotency. Such pluripotency markers may include Nanog, BMP4, FGF5, Oct4, Sox-2 and Utf1, etc

Mesenchymal stem cells made by the methods described here may be non-tumorigenic or non-teratogenic or both.

Accordingly, the mesenchymal stem cells may be such that they do not substantially induce formation of teratoma when transplanted to a recipient animal. The mesenchymal stem cells may be such that when implanted into an immune compromised or immunodeficient host animal do not result in tumours.

The recipient animal may comprise an immune compromised recipient animal. The immune compromised or immunodeficient host animal may comprise a SCID mouse or a Rag1−/− mouse. The cell may be such that a recipient animal, into which a mesenchymal stem cell is transformed, does not show teratoma formation for a suitable period of time such as 3 weeks, or 2 to 9 months, following implantation. The mesenchymal stem cells may be such that they do not form tumours after prolonged periods of implantation, such as greater than 2 weeks, for example greater than 2 months, such as greater than 9 months.

A detailed protocol for tumourigenicity testing follows.

1×10⁶ embryonic stem cells are transplanted subcutaneously into SCID mice. At three weeks when embryonic stem cell-derived tumors are about 1 cm in diameter, mesenchymal stem cells labeled with Qdot® nanocrystals (655 nm emission) using a Qtracker® Cell Labeling Kit (Quantum Dot Corp, Hayward, Calif.) are injected into the embryonic stem cell-derived teratoma.

Three days later, the mice are euthanized with an overdose of anesthesia and the tumors are removed. The tumors are fixed in 4% paraformaldehyde and cryosectioned at 20 μM thickness. The sections are assayed for pecam-1 immunoreactivity using rat anit-pecam1 (Pharmingen, San Diego, Calif.) followed by FITC-conjugated rabbit anti-rat antibody (Chemicon, Temecula, Calif.), and counterstained with DAPI. The sections are analyzed by confocal microscopy.

Mesenchymal stem cells made by the methods described here are may also display one or more of the following characteristics. The mesenchymal stem cells may be maintainable in cell culture for a prolonged period, such as greater than 40 generations. They may have a substantially stable karyotype as assessed by chromosome number, such when maintained in cell culture for at least 10 generations. They also may display a substantially stable gene expression pattern from generation to generation. By this we mean that the expression levels one or more, such as substantially all, of a chosen set of genes does not vary significantly between a mesenchymal stem cells in one generation and mesenchymal stem cells in the next generation.

The set of genes may comprise one or more, a subset, or all of, the following: cerberus (GenBank Accession nos: NM_{—}009887, AF031896, AF035579), FABP (GenBank Accession nos: NM_{—}007980, M65034, AY523818, AY523819), Foxa2 (GenBank Accession nos: NM_{—}010446, X74937, L10409), Gata-1 (GenBank Accession nos: NM_{—}008089, X15763, BC052653), Gata-4 (GenBank Accession nos: NM_{—}008092, AF179424, U85046, M98339, AB075549), Hesx1 (GenBank Accession nos: NM_{—}010420, X80040, U40720, AK082831), HNF4a (GenBank Accession nos: NM_{—}008261, D29015, BC039220), c-kit (GenBank Accession nos: NM_{—}021099, Y00864, AY536430, BC075716, AK047010, BC026713, BC052457, AK046795), PDGFRα (NM_{—}011058, M57683, M84607, BC053036), Oct4 (GenBank Accession nos: NM_{—}013633, X52437, M34381, BC068268), Runx1 (GenBank Accession nos: NM_{—}009821, D26532, BC069929, AK051758), Sox17 (GenBank Accession nos: NM_{—}011441, D49474, L29085, AK004781), Sox2 (GenBank Accession nos: NM_{—}011443, U31967, AB108673), Brachyury (NM_{—}009309, X51683), TDGF1 (GenBank Accession nos: NM_{—}011562, M87321) and Tie-2 (GenBank Accession nos: NM_{—}013690, X67553, X71426, D13738, BC050824).

The methods described here enable the production of mesenchymal stem cells as well as differentiated cells, which comprise clonal descendants of mesenchymal stem cells. The term “clonal descendant” of a cell refers to descendants of the cells which have not undergone substantially any transforming treatment or genetic alteration. Such clonal descendants have not undergone substantial genomic changes are substantially genetically identical to the parent cell, or an ancestor, such as the mesenchymal stem cell. The term “mesenchymal stem cells” should also be taken to include cell lines derived from mesenchymal stem cells, i.e., mesenchymal stem cell lines, and vice versa.

### Long-Term Maintenance in Culture

The methods described here involve culturing the mesenchymal stem cells or their descendants for more than one generation.

For example, the cells may be cultured for more than 5, more than 10, more than 15, more than 20, more than 25, more than 50, more than 40, more than 45, more than 50, more than 100, more than 200, more than 500 or more than 800 generations. In particular, the cell lines may be maintained for 1, 2, 3, 4, 5, 6, 7, 8, 9, 10, 11, 12, 13, 14, 15, 20, 25, 30, 35, 40, 45, 50, 75, 100, 200, 500 or more generations.

Cells in culture will generally continue growing until confluence, when contact inhibition causes cessation of cell division and growth. Such cells may then be dissociated from the substrate or flask, and “split” or passaged, by dilution into tissue culture medium and replating. The cultured cells may therefore be passaged, or split during culture. They may be split at a ratio of 1:2 or more, such as 1:3 or 1:4, 1:5 or more. The term “passage” designates the process consisting in taking an aliquot of a confluent culture of a cell line, in inoculating into fresh medium, and in culturing the line until confluence or saturation is obtained.

The mesenchymal stem cells derived according to the methods described here may however be maintained for a large number of generations. On the other hand, it has been established that “normal” (i.e., untransformed somatic) cells derived directly from an organism are not immortal. In other words, such somatic cells have a limited life span in culture (they are mortal). They will not continue growing indefinitely, but will ultimately lose the ability to proliferate or divide after a certain number of generations. On reaching a “crisis phase” such cells die after about 50 generations. Thus, such somatic cells may only be passaged a limited number of times.

The mesenchymal stem cells described here are able to maintain self-renewal without the requirement for transformation. Thus, for example, known transformation treatments such as fusion with immortalised cells such as tumour cells or tumour cell lines, viral infection of a cell line with tranforming viruses such as SV40, EBV, HBV or HTLV-1, transfection with specially adapted vectors, such as the SV40 vector comprising a sequence of the large T antigen (R. D. Berry et al., Br. J. Cancer, 57, 287-289, 1988), telomerase (Bodnar-A-G. et. al., Science (1998) 279: p. 349-52) or a vector comprising DNA sequences of the human papillomavirus (U.S. Pat. No. 5,376,542), introduction of a dominant oncogene, or by mutation are therefore not required in the methods described here for making mesenchymal stem cell lines.

According to the methods described here, mesenchymal stem cells may be propagated without transformation for more than 50 generations. In some embodiments, the mesenchymal stem cells may be propagated indefinitely and without transformation as mesenchymal stem cell lines. The mesenchymal stem cells and mesenchymal stem cells lines may be lineage restricted. In particular, they may be such that they are not capable of giving rise to all three germ layers. In some embodiments, the mesenchymal stem cells lines may be non-pluripotent.

We further provide a composition comprising a plurality of cells, wherein a majority of the cells are mesenchymal stem cells. For example, at least 60% of the cells may comprise mesenchymal stem cells. In addition, we provide an isolated mesenchymal stem cell. The term cell line may be taken to refer to cells that can be maintained and grown in culture and display an immortal or indefinite life span.

### Diseases Treatable

The proteome of mesenchymal stem cells obtained by the methods described here may be analysed.

The mesenchymal stem cells thus obtained my have an expression profile which is similar to that of mesenchymal stem cells, such as hESC-MSCs described in WO2007/027157 or WO2007/027156. Thus, mesenchymal stem cells derived by our methods may have significant biological similarities to their counterparts, e.g., in their ability to secrete paracrine factors. Accordingly, the mesenchymal stem cells described here may be used for any purpose for which hESC-MSCs such as those described in WO2007/027157 or WO2007/027156 are suitable.

The pre-natal mesenchymal stem cells may be used to treat diseases of metabolism, defense response, and tissue differentiation including vascularization, hematopoiesis and skeletal development, or whose repair or treatment involves any one or more of these biological processes. Similarly, the proteins expressed, such as secreted, by the pre-natal mesenchymal stem cells, singly or in combination, such as in the form of a conditioned medium, may be used to supplement the activity of, or in place of, the pre-natal mesenchymal stem cells, for the purpose of for example treating or preventing such diseases.

The pre-natal mesenchymal stem cells may be used to activate signalling pathways in cardiovascular biology, bone development and hematopoiesis such as Jak-STAT, MAPK, Toll-like receptor, TGF-beta signalling and mTOR signaling pathways. Thus, the pre-natal mesenchymal stem cells, proteins expressed by them, etc, may be used to prevent or treat a disease in which any of these signalling pathways is involved, or whose aetiology involves one or more defects in any one or more of these signalling pathways.

Accordingly, pre-natal mesenchymal stem cells may be used to treat cardiac failure, bone marrow disease, skin disease, burns and degenerative diseases such as diabetes, Alzheimer's disease, Parkinson's disease and cancer.

Pre-natal mesenchymal stem cells may also be used to treat myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

They may be used to aid wound healing, scar reduction, bone formation, a bone graft or bone marrow transplantation in an individual.

We provide for the use of mesenchymal stem cells obtained herein or medium conditioned by such mesenchymal stem cells in the regulation of pathways including any one or more of the following: cytoskeletal regulation by Rho GTPase, cell cycle, integrin signalling pathway, Inflammation mediated by chemokine & cytokine signaling pathway, FGF signaling pathway, EGF receptor signaling pathway, angiogenesis, plasminogen activating cascade, blood coagulation, glycolysis, ubiquitin proteasome pathway, de novo purine biosynthesis, TCA cycle, phenylalanine biosynthesis, heme biosynthesis.

We provide also for the use of mesenchymal stem cells obtained herein or medium conditioned by such mesenchymal stem cells in the regulation of processes including any one or more of the following: cell structure and motility, cell structure, cell communication, cell motility, cell adhesion, endocytosis, mitosis, exocytosis, cytokinesis, cell cycle, immunity and defense, cytokine/chemokine mediated immunity, macrophage-mediated immunity, granulocyte-mediated immunity, ligand-mediated signaling, cytokine and chemokine mediated signaling pathway, signal transduction, extracellular matrix protein-mediated signaling, growth factor homeostasis, receptor protein tyrosine kinase signaling pathway, cell adhesion-mediated signaling, cell surface receptor mediated signal transduction, JAK-STAT cascade, antioxidation and free radical removal, homeostasis, stress response, blood clotting, developmental processes, mesoderm development, skeletal development, angiogenesis, muscle development, muscle contraction, protein metabolism and modification, proteolysis, protein folding, protein complex assembly, amino acid activation, intracellular protein traffic, other protein targeting and localization, amino acid metabolism, protein biosynthesis, protein disulfide-isomerase reaction, carbohydrate metabolism, glycolysis, pentose-phosphate shunt, other polysaccharide metabolism, purine metabolism, regulation of phosphate metabolism, vitamin metabolism, amino acid biosynthesis, pre-mRNA processing, translational regulation, mRNA splicing.

We further provide for the use of mesenchymal stem cells obtained herein or medium conditioned by such mesenchymal stem cells in the supply of functions including any one or more of the following: signaling molecule, chemokine, growth factor, cytokine, interleukin, other cytokine, extracellular matrix, extracellular matrix structural protein, other extracellular matrix, extracellular matrix glycoprotein, protease, metalloprotease, other proteases, protease inhibitor, metalloprotease inhibitor, serine protease inhibitor, oxidoreductase, dehydrogenase, peroxidase, chaperone, chaperonin, Hsp 70 family chaperone, other chaperones, synthetase, synthase and synthetase, select calcium binding protein, aminoacyl-tRNA synthetase, lyase, isomerase, other isomerase, ATP synthase, hydratase, transaminase, other lyase, other enzyme regulator, select regulatory molecule, actin binding cytoskeletal protein, cytoskeletal protein, non-motor actin binding protein, actin and actin related protein, annexin, tubulin, cell adhesion molecule, actin binding motor protein, intermediate filament, ribonucleoprotein, ribosomal protein, translation factor, other RNA-binding protein, histone, calmodulin related protein, vesicle coat protein.

Furthermore, the mesenchymal stem cells obtained herein or medium conditioned by such mesenchymal stem cells may be used to treat diseases which these functions may have a role in, or whose repair or treatment involves any one or more of these biological processes. Similarly, the proteins expressed by the mesenchymal stem cells, singly or in combination, such as in the form of a conditioned medium, may be used to supplement the activity of, or in place of, the mesenchymal stem cells, for the purpose of for example treating or preventing such diseases.

The gene products expressed by the mesenchymal stem cells obtained herein may be used to activate important signalling pathways in cardiovascular biology, bone development and hematopoiesis such as Jak-STAT, MAPK, Toll-like receptor, TGF-beta signalling and mTOR signaling pathways. Accordingly, the hESC-MSCs, proteins expressed by them, etc, may be used to prevent or treat a disease in which any of these signalling pathways is involved, or whose aetiology involves one or more defects in any one or more of these signalling pathways.

Accordingly, such a conditioned medium may be used to treat cardiac failure, bone marrow disease, skin disease, burns and degenerative diseases such as diabetes, Alzheimer's disease, Parkinson's disease and cancer.

Such a conditioned medium may also be used to treat myocardial infarction, a cutaneous wound, a dermatologic disorder, a dermatological lesion, dermatitis, psoriasis, condyloma, verruca, hemangioma, keloid, skin cancer, atopic dermatitis, Behcet disease, chronic granulomatous disease, cutaneous T cell lymphoma, ulceration, a pathological condition characterised by initial injury inducing inflammation and immune dysregulation leading to chronic tissue remodeling including fibrosis and loss of function, renal ischemic injury, cystic fibrosis, sinusitis and rhinitis or an orthopaedic disease.

The conditioned medium may be used to aid wound healing, scar reduction, bone formation, a bone graft or bone marrow transplantation in an individual.

In particular, the conditioned medium may be used to regulate the processes involved in vascularisation, hematology (specifically immune processes) or musculoskeletal development, etc.

Such a composition may be used for any purpose the conditioned medium may be used. Unless the context dictates otherwise, the term “conditioned medium” should be taken to include not only cell culture medium exposed to MSCs as well as such a composition comprising one or more, such as substantially all, the polypeptides which are present in the conditioned medium.

Furthermore, any one or more proteins secreted from the mesenchymal stem cells described here, including in the form of conditioned media, may be used for the same purposes as the hESC-MSCs as described in WO2007/027157 or WO2007/027156.

The pre-natal mesenchymal stem cells may also be used as sources for any of the proteins secreted or expressed by them. We therefore provide for a method of producing a polypeptide comprising obtaining a mesenchymal stem cell as described, culturing the mesenchymal stem cell and isolating the polypeptide from the mesenchymal stem cell, such as from a medium in which the mesenchymal stem cell is growing.

Dermatologic Disorders

Media conditioned by the pre-natal mesenchymal stem cells obtained by the methods described in this document may be used for the treatment of dermatologic disorders.

Therefore the topical application of such conditioned medium on cutaneous wounds or dermatological lesions or disorders such as dermatitis or psoriasis improves healing and reduces scarring. It should also maintain homeostasis. The conditioned medium may be delivered in liposome-based emulsion, gel or cream formulations and part of standard wound dressing. The conditioned medium may also be used as a supplement in cosmetic skincare product to promote skin repair and healing.

A suitable animal model to test the efficacy of such conditioned medium on cutaneous disorders is a mouse model of dermatitis. Epicutaneous sensitization of mice is performed as described earlier [A72, A73]. Briefly, 50 μg of Blo t 5 in 100 μl of PBS or PBS alone are applied to 1 cm² gauze and patched to the skin with a transparent dressing (Smith Nephew). This procedure is repeated twice over a period of 50 days. CM and NCM are then applied to 1 cm2 gauze and patched to the skin as described above for varying period of time.

For histological examination of skin inflammation, specimens are obtained from the patched skins and fixed in 10% buffered neutral formalin immediately.

Asthma and Allergy

Media conditioned by the pre-natal mesenchymal stem cells obtained by the methods described in this document may be used for the treatment of asthma and allergy.

Asthma is a complex disease with an equally complex etiology caused by a poorly characterized set of genetic and environmental factors. The resulting pathology is immune dysregulation leading to chronic inflammation of the airways and subepithelial fibrosis characterized by increase in smooth muscle mass and increased deposition of extracellular matrix proteins and subsequently, reduced lung function.

Current therapies include modulating several factors or signaling pathways e.g. the ECM, integrins, and mesenchymal cell function [A74], toll-like receptors [A75], growth factors such as TGF-β and EGF [A76, A77] and the IL6 pathway [A78]. The CM by hESC-MSCs has been predicted to have biological effects on these targeted area and we predict that the CM helps restore immune regulation in asthmatic lungs and promote tissue repair and minimize scarring of lung tissues.

To investigate the effects of such conditioned medium (CM) and the non-conditioned medium (NCM) on chronic airway inflammation and airway, epicutaneous sensitization of mice are performed as described above [A72, A73] After 50 days, the patched mice are anesthetized and receive intranasal challenge with 50 μg of Blo t 5 for three consecutive days. Twenty-four hours after the last dose, airway hyperresponsivensess (AHR) is measured using invasive BUXCO [A79]. The mice are anesthetized and given conditioned medium or non-conditioned medium intranasally for three consecutive days. Twenty-four hours after the last dose, airway hyperresponsivensess (AHR) is measured. BAL fluid is collected after another twenty-four hours. Following bronchoalveolar lavage collection, lungs are fixed with 10% neutralized formalin.

To investigate the effects of CM and the NCM on acute airway inflammation and airway.

A Blot t 5 specific Th2 cell line which secretes high level of IL-4, IL-5, IL-13 and with undetectable level of IFN-γ, is used to establish a mouse allergy model. Briefly, sensitization of naïve mice is done by adoptive transfer of 2.5×10⁶ Blo t 5 specific Th2 cells intravenously in each mouse. These mice are anesthetized and intranasal (IN) challenged with 50 μg of Blo t 5 for three consecutive days. Twenty-four hours after the last IN challenge, airway hyperresponsivensess (AHR) is measured using invasive BUXCO [A79]. The mice are then anesthetized and given CM or NCM intranasally for three consecutive days. BAL fluid is collected at forty-eight hours after the last Blo t 5 challenge. Following bronchoalveolar lavage collection, lungs are fixed with 10% neutralized formalin for histopathological analysis.

In clinical practice, the use of CM to treat lung disease may be administered effectively using standard aerosol therapy [A80-86].

Other Diseases

Media conditioned by the pre-natal mesenchymal stem cells obtained by the methods described in this document may be used for the treatment of other diseases.

In general, we predict that such conditioned medium is useful in restoring homeotstasis and promoting tissue repair in pathological conditions where the initial injury induced inflammation and immune dysregulation leads to chronic tissue remodeling that includes fibrosis and loss of function. Other diseases include renal ischemic injury, cystic fibrosis, sinusitis and rhinitis.

Orthopedics

Media conditioned by the pre-natal mesenchymal stem cells obtained by the methods described in this document may be used for the treatment of orthopedic disorders.

Current therapeutic strategies for repair of musculoskeletal tissue often include the use of a biomaterial (ceramics or polymers) not only to provide mechanical support but also as a scaffold to promote cell migration, cell adhesion, proliferation and differentiation to initiate vascularization and ultimately new bone formation [A87-90]. Based on the computation analysis of such conditioned medium, incorporation of CM into the scaffold design may enhance cell migration, proliferation, adhesion, skeletal differentiation and vascularization of the scaffold.

To test the effect of CM on bone regeneration in defects that would otherwise have led to atrophic nonunions, New Zealand white rabbits receive a 15-mm critical size defect on one radius [A91], which is filled with a suitable matrix such as a collagen sponge or hydrogel coated with either CM or NCM. Radiographs are obtained every 3 weeks. After 6 or 12 weeks, animals are killed. New bone is measured by microCT scans and vascularity is measured using anti-CD31 staining of endothelial cells in the implant. There should be increased vascularity at the least initially and also increased new bone formation.

To test the effects of CM on cartilage repair, a rabbit model of osteochondral injury [A92] is used. CM is coated on a suitable scaffold such as collagen or hydrogel and implanted into 3-mm osteochondral knee defects [A93]. For clinical applications, CM may be used by incorporating the CM into existing scaffolds or bone grafts [A94].

Bone Marrow Transplantation

MSCs have been shown to enhance bone marrow transplantation [A95] and ameliorate graft versus host disease. Transplantation of MSCs has been shown to improve the outcome of allogeneic transplantation by promoting hematopoietic engraftment [A96] and limiting GVHD [A97, A98]. It is postulated that MSCs mediate these effects through the enhancement of the hematopoieitic stem cell niche [A99] and the induction of tolerance and reduce GVHD, rejection of allogeneic tissue transplant and modulation of inflammation [A98], possiblly through the secretion of soluble factors [A100].

Potential clinical applications of such conditioned medium by MSCs: expansion of hematopoietic stem cell population in vitro by supplementing culture media with CM or in vivo by infusing CM with hematopoietic stem cells during transplantation, ameliorate GVHD by intravenous infusion of CM or induction of immune tolerance to transplanted cells or tissues by intravenous infusion of CM as part of the pre and post-transplant therapy.

### Heart Disease

The mesenchymal stem cells described here may be used for treatment or prevention of heart disease.

Heart disease is an umbrella term for a variety for different diseases affecting the heart. As of 2007, it is the leading cause of death in the United States, England, Canada and Wales, killing one person every 34 seconds in the United States alone. Heart disease includes any of the following.

Coronary Heart Disease

Coronary artery disease is a disease of the artery caused by the accumulation of atheromatous plaques within the walls of the arteries that supply the myocardium. Angina pectoris (chest pain) and myocardial infarction (heart attack) are symptoms of and conditions caused by coronary heart disease. Over 459,000 Americans die of coronary heart disease every year. In the United Kingdom, 101,000 deaths annually are due to coronary heart disease.

Cardiomyopathy

Cardiomyopathy is the deterioration of the function of the myocardium (i.e., the actual heart muscle) for any reason. People with cardiomyopathy are often at risk of arrhythmia and/or sudden cardiac death. Extrinsic cardiomyopathies—cardiomyopathies where the primary pathology is outside the myocardium itself comprise the majority of cardiomyopathies. By far the most common cause of a cardiomyopathy is ischemia.

The World Health Organization includes as specific cardiomyopathies: Alcoholic cardiomyopathy, coronary artery disease, congenital heart disease, nutritional diseases affecting the heart, ischemic (or ischaemic) cardiomyopathy, hypertensive cardiomyopathy, valvular cardiomyopathy, inflammatory cardiomyopathy.

Also included are:

Cardiomyopathy secondary to a systemic metabolic disease

Intrinsic cardiomyopathies (weakness in the muscle of the heart that is not due to an identifiable external cause)

Dilated cardiomyopathy (DCM, the most common form, and one of the leading indications for heart transplantation. In DCM the heart (especially the left ventricle) is enlarged and the pumping function is diminished)

Hypertrophic cardiomyopathy (HCM or HOCM, a genetic disorder caused by various mutations in genes encoding sarcomeric proteins. In HCM the heart muscle is thickened, which can obstruct blood flow and prevent the heart from functioning properly),

Arrhythmogenic right ventricular cardiomyopathy (ARVC, which arises from an electrical disturbance of the heart in which heart muscle is replaced by fibrous scar tissue. The right ventricle is generally most affected)

Restrictive cardiomyopathy (RCM, which is the least common cardiomyopathy. The walls of the ventricles are stiff, but may not be thickened, and resist the normal filling of the heart with blood).

Noncompaction Cardiomyopathy—the left ventricle wall has failed to properly grow from birth and such has a spongy appearance when viewed during an echocardiogram.

Cardiovascular Disease

Cardiovascular disease is any of a number of specific diseases that affect the heart itself and/or the blood vessel system, especially the veins and arteries leading to and from the heart. Research on disease dimorphism suggests that women who suffer with cardiovascular disease usually suffer from forms that affect the blood vessels while men usually suffer from forms that affect the heart muscle itself. Known or associated causes of cardiovascular disease include diabetes mellitus, hypertension, hyperhomocysteinemia and hypercholesterolemia.

Types of cardiovascular disease include atherosclerosis

Ischaemic Heart Disease

Ischaemic heart disease is disease of the heart itself, characterized by reduced blood supply to the organs. This occurs when the arteries that supply the oxygen and the nutrients gets stopped and the heart will not get enough of the oxygen and the nutrients and will eventually stop beating.

Heart Failure

Heart failure, also called congestive heart failure (or CHF), and congestive cardiac failure (CCF), is a condition that can result from any structural or functional cardiac disorder that impairs the ability of the heart to fill with or pump a sufficient amount of blood throughout the body. Cor pulmonale is a failure of the right side of the heart.

Hypertensive Heart Disease

Hypertensive heart disease is heart disease caused by high blood pressure, especially localised high blood pressure. Conditions that can be caused by hypertensive heart disease include: left ventricular hypertrophy, coronary heart disease, (Congestive) heart failure, hypertensive cardiomyopathy, cardiac arrhythmias, inflammatory heart disease, etc.

Inflammatory heart disease involves inflammation of the heart muscle and/or the tissue surrounding it. Endocarditis comprises inflammation of the inner layer of the heart, the endocardium. The most common structures involved are the heart valves. Inflammatory cardiomegaly. Myocarditis comprises inflammation of the myocardium, the muscular part of the heart.

Valvular Heart Disease

Valvular heart disease is disease process that affects one or more valves of the heart. The valves in the right side of the heart are the tricuspid valve and the pulmonic valve. The valves in the left side of the heart are the mitral valve and the aortic valve. Included are aortic valve stenosis, mitral valve prolapse and valvular cardiomyopathy.

[The above text is adapted from Heart disease. (2009 Feb. 3). In Wikipedia, The Free Encyclopedia. Retrieved 06:33, Feb. 20, 2009, from http://en.wikipedia.org/w/index.php?title=Heart_disease&oldid=268290924]

### Regulators of Mesenchymal Stem Cell Formation

Our methods may also be used to identify putative regulators of mesenchymal stem cell formation from embryonic stem cells. The methods involve conducting the methods described for production of mesenchymal stem cells in the presence and absence of a candidate molecule, and identifying if the presence of the molecule has any effect on the process. For example, a molecule which accelerates the production of mesenchymal stem cells may be used as a positive regulator of mesenchymal stem cell formation. Conversely, a molecule which retards the process can be considered an inhibitor of mesenchymal stem cell formation.

We also provide a cell, such as a mesenchymal stem cell, obtainable according to the method. Preparations of mesenchymal stem cells may be either too impure, or not substantially phenotypically similar or identical (e.g., with respect to gene expression), or may not be suitable for clinical purposes if they are produced by methods involving co-culture or presence of serum. With culture according to the invention, this can give rise to substantially 100% pure preparations of mesenchymal stem cells which are similar or identical (such as homogenous) to each other.

In addition, we describe a process for producing differentiated cells, the method comprising obtaining a mesenchymal stem cell by a method as described herein, and differentiating the mesenchymal stem cell. For example, we provide for methods of differentiating to adipocytes, chondrocytes and osteocytes, etc. We further provide differentiated cells obtainable by such methods. Cell lines made from such mesenchymal stem cells and differentiated cells are also provided. The term cell line may refer to cells that can be maintained and grown in culture and display an immortal or indefinite life span.

### Uses

The methods and compositions described here may be used for up-regulating expression of mesenchymal or endothelial markers of a cell. They may also or instead be used for down-regulating expression of stem cell or pluripotency markers of a cell. The methods and compositions described here may be used to identify an agent capable of promoting or retarding self-renewal or differentiation of a stem cell. Such a method comprises performing a method according to any preceding claim in the presence of a candidate molecule, and determining an effect thereon.

The methods and compositions described here may also be used for the production of a differentiated cell for the treatment of, or the preparation of a pharmaceutical composition for the treatment of, any one of the following: a disease treatable by regenerative therapy, cardiac failure, bone marrow disease, skin disease, burns, degenerative disease such as diabetes, Alzheimer's disease, Parkinson's disease and cancer.

Mesenchymal stem cells and differentiated cells made according to the methods described here can be used for a variety of commercially important research, diagnostic, and therapeutic purposes. These uses are generally well known in the art, but will be described briefly here.

For example, stem cells may be used to generate mesenchymal stem cells and differentiated cell populations for regenerative therapy. Mesenchymal stem cells and differentiated cells may be made by ex vivo expansion or directly administered into a patient. They may also be used for the re-population of damaged tissue following trauma.

Thus, adipocytes or fat tissues may therefrom may be used to fill up cavities or depressions during reconstructive or plastic surgery. Chondrocytes may be used for cartilage repair while osteocytes may be used for bone repair. The mesenchymal stem cells made by the methods and compositions described here may be differentiated into any of these cell types and used for the purposes described.

Mesenchymal stem cells and differentiated cells produced by the methods described in this document may be used for the treatment of degenerative diseases such as diabetes, Alzheimer's disease, Parkinson's disease, etc. Stem cells, for example may be used as sources of mesenchymal stem cells and differentiated cells for NK or dendritic cells for immunotherapy for cancer, which mesenchymal stem cells and differentiated cells may be made by the methods and compositions described here.

It will be evident that the methods and compositions described here enable the production of mesenchymal stem cells, which may of course be made to differentiate using methods known in the art. Thus, any uses of differentiated cells will equally attach to those mesenchymal stem cells for which they are sources.

Mesenchymal stem cells and differentiated cells produced by the methods and compositions described here may be used for, or for the preparation of a pharmaceutical composition for, the treatment of a disease. Such disease may comprise a disease treatable by regenerative therapy, including cardiac failure, bone marrow disease, skin disease, burns, degenerative disease such as diabetes, Alzheimer's disease, Parkinson's disease, etc and cancer.

### Differentiated Cells

Differentiated cells, such as terminally differentiated cells, may be derived from the mesenchymal stem cells or cell lines made according to the methods described. We therefore disclose methods for generating differentiated cells, the methods comprising generating mesenchymal stem cells or cell lines as described, and deriving differentiated cells from these. The mesenchymal stem cells made by the methods and compositions described here may be differentiated into any of these cell types and used for the purposes described.

Differentiated cells which may be made according to the methods described here may include any or all of the following:

i) adipocyte: the functional cell type of fat, or adipose tissue, that is found throughout the body, particularly under the skin. Adipocytes store and synthesize fat for energy, thermal regulation and cushioning against mechanical shock

ii) cardiomyocytes: the functional muscle cell type of the heart that allows it to beat continuously and rhythmically

iii) chondrocyte: the functional cell type that makes cartilage for joints, ear canals, trachea, epiglottis, larynx, the discs between vertebrae and the ends of ribs

iv) fibroblast: a connective or support cell found within most tissues of the body. Fibroblasts provide an instructive support scaffold to help the functional cell types of a specific organ perform correctly.

v) hepatocyte: the functional cell type of the liver that makes enzymes for detoxifying metabolic waste, destroying red blood cells and reclaiming their constituents, and the synthesis of proteins for the blood plasma

vi) hematopoietic cell: the functional cell type that makes blood. Hematopoietic cells are found within the bone marrow of adults. In the fetus, hematopoietic cells are found within the liver, spleen, bone marrow and support tissues surrounding the fetus in the womb.

vii) myocyte: the functional cell type of muscles

viii) neuron: the functional cell type of the brain that is specialized in conducting impulses

ix) osteoblast: the functional cell type responsible for making bone

x) islet cell: the functional cell of the pancreas that is responsible for secreting insulin, glucogon, gastrin and somatostatin. Together, these molecules regulate a number of processes including carbohydrate and fat metabolism, blood glucose levels and acid secretions into the stomach.

### Uses of Mesenchymal Stem Cells and Differentiated Cells

Mesenchymal stem cells and differentiated cells made according to the methods and compositions described here may be used for a variety of commercially important research, diagnostic, and therapeutic purposes.

For example, populations of differentiated cells may be used to prepare antibodies and cDNA libraries that are specific for the differentiated phenotype. General techniques used in raising, purifying and modifying antibodies, and their use in immunoassays and immunoisolation methods are described in Handbook of Experimental Immunology (Weir & Blackwell, eds.); Current Protocols in Immunology (Coligan et al., eds.); and Methods of Immunological Analysis (Masseyeff et al., eds., Weinheim: VCH Verlags GmbH). General techniques involved in preparation of mRNA and cDNA libraries are described in RNA Methodologies: A Laboratory Guide for Isolation and Characterization (R. E. Farrell, Academic Press, 1998); cDNA Library Protocols (Cowell & Austin, eds., Humana Press); and Functional Genomics (Hunt & Livesey, eds., 2000). Relatively homogeneous cell populations are particularly suited for use in drug screening and therapeutic applications.

These and other uses of mesenchymal stem cells and differentiated cells are described in further detail below, and elsewhere in this document. The mesenchymal stem cells and differentiated cells may in particular be used for the preparation of a pharmaceutical composition for the treatment of disease. Such disease may comprise a disease treatable by regenerative therapy, including cardiac failure, bone marrow disease, skin disease, burns, degenerative disease such as diabetes, Alzheimer's disease, Parkinson's disease, etc and cancer.

As shown in the Examples, the mesenchymal stem cells made by the methods and compositions described here have similar or identical properties to human embryonic stem cell derived mesenchymal stem cells (hESC-MSCs), for example as described in WO2007/027157 or WO2007/027156. Therefore, the mesenchymal stem cells, and any differentiated cells made from these, may be used in any of the applications for which hESC-mesenchymal stem cells are known to be used, or in which it is possible for them to be used.

### Drug Screening

Mesenchymal stem cells and differentiated cells made according to the methods and compositions described here may also be used to screen for factors (such as solvents, small molecule drugs, peptides, polynucleotides, and the like) or environmental conditions (such as culture conditions or manipulation) that affect the characteristics of mesenchymal stem cells or differentiated cells.

In some applications, mesenchymal stem cells and differentiated cells are used to screen factors that promote maturation, or promote proliferation and maintenance of such cells in long-term culture. For example, candidate maturation factors or growth factors are tested by adding them to mesenchymal stem cells or differentiated cells in different wells, and then determining any phenotypic change that results, according to desirable criteria for further culture and use of the cells.

Furthermore, gene expression profiling of mesenchymal stem cells and differentiated cells may be used to identify receptors, transcription factors, and signaling molecules that are unique or highly expressed in these cells. Specific ligands, small molecule inhibitors or activators for the receptors, transcription factors and signaling molecules may be used to modulate differentiation and properties of mesenchymal stem cells and differentiated cells.

Particular screening applications relate to the testing of pharmaceutical compounds in drug research. The reader is referred generally to the standard textbook “In vitro Methods in Pharmaceutical Research”, Academic Press, 1997, and U.S. Pat. No. 5,030,015), as well as the general description of drug screens elsewhere in this document. Assessment of the activity of candidate pharmaceutical compounds generally involves combining the differentiated cells with the candidate compound, determining any change in the morphology, marker phenotype, or metabolic activity of the cells that is attributable to the compound (compared with untreated cells or cells treated with an inert compound), and then correlating the effect of the compound with the observed change.

The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug—drug interaction effects. In some applications, compounds are screened initially for potential toxicity (Castell et al., pp. 375-410 in “In vitro Methods in Pharmaceutical Research,” Academic Press, 1997). Cytotoxicity can be determined in the first instance by the effect on cell viability, survival, morphology, and expression or release of certain markers, receptors or enzymes. Effects of a drug on chromosomal DNA can be determined by measuring DNA synthesis or repair. [³H]thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to A. Vickers (PP 375-410 in “In vitro Methods in Pharmaceutical Research,” Academic Press, 1997) for further elaboration.

### Tissue Regeneration

Mesenchymal stem cells and differentiated cells made according to the methods and compositions described here may also be used for tissue reconstitution or regeneration in a human patient in need thereof. The cells are administered in a manner that permits them to graft to the intended tissue site and reconstitute or regenerate the functionally deficient area.

For example, the methods and compositions described here may be used to modulate the differentiation of stem cells. Mesenchymal stem cells and differentiated cells may be used for tissue engineering, such as for the growing of skin grafts. Modulation of stem cell differentiation may be used for the bioengineering of artificial organs or tissues, or for prosthetics, such as stents.

### Cancer

Mesenchymal stem cells and differentiated cells made by the methods and compositions described here may be used for the treatment of cancer.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia.

More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer. Further examples are solid tumor cancer including colon cancer, breast cancer, lung cancer and prostrate cancer, hematopoietic malignancies including leukemias and lymphomas, Hodgkin's disease, aplastic anemia, skin cancer and familiar adenomatous polyposis. Further examples include brain neoplasms, colorectal neoplasms, breast neoplasms, cervix neoplasms, eye neoplasms, liver neoplasms, lung neoplasms, pancreatic neoplasms, ovarian neoplasms, prostatic neoplasms, skin neoplasms, testicular neoplasms, neoplasms, bone neoplasms, trophoblastic neoplasms, fallopian tube neoplasms, rectal neoplasms, colonic neoplasms, kidney neoplasms, stomach neoplasms, and parathyroid neoplasms. Breast cancer, prostate cancer, pancreatic cancer, colorectal cancer, lung cancer, malignant melanoma, leukaemia, lympyhoma, ovarian cancer, cervical cancer and biliary tract carcinoma are also included.

The mesenchymal stem cells and differentiated cells made according to the methods and compositions described here may also be used in combination with anticancer agents such as endostatin and angiostatin or cytotoxic agents or chemotherapeutic agent. For example, drugs such as such as adriamycin, daunomycin, cis-platinum, etoposide, taxol, taxotere and alkaloids, such as vincristine, and antimetabolites such as methotrexate. The term “cytotoxic agent” as used herein refers to a substance that inhibits or prevents the function of cells and/or causes destruction of cells. The term is intended to include radioactive isotopes (e.g. I, Y, Pr), chemotherapeutic agents, and toxins such as enzymatically active toxins of bacterial, fungal, plant or animal origin, or fragments thereof.

Also, the term includes oncogene product/tyrosine kinase inhibitors, such as the bicyclic ansamycins disclosed in WO 94/22867; 1,2-bis(arylamino) benzoic acid derivatives disclosed in EP 600832; 6,7-diamino-phthalazin-1-one derivatives disclosed in EP 600831; 4,5-bis(arylamino)-phthalimide derivatives as disclosed in EP 516598; or peptides which inhibit binding of a tyrosine kinase to a SH2-containing substrate protein (see WO 94/07913, for example). A “chemotherapeutic agent” is a chemical compound useful in the treatment of cancer. Examples of chemotherapeutic agents include Adriamycin, Doxorubicin, 5-Fluorouracil (5-FU), Cytosine arabinoside (Ara-C), Cyclophosphamide, Thiotepa, Busulfan, Cytoxin, Taxol, Methotrexate, Cisplatin, Melphalan, Vinblastine, Bleomycin, Etoposide, Ifosfamide, Mitomycin C, Mitoxantrone, Vincristine, VP-16, Vinorelbine, Carboplatin, Teniposide, Daunomycin, Caminomycin, Aminopterin, Dactinomycin, Mitomycins, Nicotinamide, Esperamicins (see U.S. Pat. No. 4,675,187), Melphalan and other related nitrogen mustards, and endocrine therapies (such as diethylstilbestrol (DES), Tamoxifen, LHRH antagonizing drugs, progestins, anti-progestins etc).

### EXAMPLES

### Example 1

### Derivation of Fetal Tissue Derived-MSCs

The collection of fetal tissue is carried out under a KK Women's and Children's Hospital (KKH) IRB approved protocol (EC200804062) in accordance with guidelines from Singapore Bioethics Advisory Committee²⁷ which stated that the decision to donate the fetal tissue must be made independently from any decision to abort.

Only patients who have already consented to Termination of Pregnancy (TOP) in KKH Outpatient Clinic are recruited. Recruitment is carried out in strict adherence to KKH IRB's regulations to ensure patient's rights and privacy, and to provide confidential counselling for patient's fully informed consent to voluntary donation. TOP for fetal abnormalities, sexual assault cases and in minor (16 and below) are excluded. Patients with medical problems are also excluded.

The aborted specimens are collected in special sterile plastic bottles and sent to the hospital Department of Laboratory Medicine for full pathological examinations. Appropriate pieces of fetal tissues are dissected, washed several times in sterile saline, minced, and placed in DMEM medium supplemented with 10% serum replacement medium, EGF (20 ηg/ml) and FGF2 (20 ηg/ml) to attach to plastic tissue culture dishes for 24-48 hours. Under this condition, MSCs whose defining characteristic is adherence to plastic migrated out of the tissues and adhere to the plastic culture dish.

The large tissue pieces are then washed off leaving a homogenous cell culture. The cells are maintained at 25-80% confluency or 15-50,000 cells per cm² and are split 1:4 at confluency by trypsinization. On reaching 2×10⁷ cells, the culture is designated P1.

Differentiation of the fetal MSCs to adipocytes, chondrocytes and osteocytes is performed using adipogenic, chondrogenic and osteogenic hMSC Differentiation BulletKits, respectively (Lonza, Walkersville, Md.) as per manufacturer' instructions. Karyotyping by G-banding is performed at the Cytogenetics Laboratory, KKH.

### Example 2

### Telomerase Activity

Relative telomerase activity is measured by SYBR® Green real time quantitative telomeric repeat amplification protocol assay using a modified method as described by Wege H. et al²⁸.

Briefly, 3 million cells are harvested and cell lysate is prepared using a commercially available mammalian cell extraction kit (Cat K269-500-1, BioVision, Singapore). The composition of the reagents for the PCR amplification is 1 μg of protein cell lysate, 10 μl of 2× SYBR Green Super Mix (Cat 170-8880, BioRad, Singapore) with 0.1 μg of TS primer (5′-AATCCGTCGAGCAGACTT-3′), 0.1 μg of ACX primer (5′-GCGCGG[CTTACC]3CTAACC-3′) and 10 mM EGTA in a total volume of 25 μl. The reaction is first incubated at 25° C. for 20 min to allow the telomerase in the cell lysate to elongate the TS primers followed by 2 min incubation at 95° C. to inactivate telomerase activity and denature the primers. The telomerase product is amplified by PCR for 40 cycles of 95° C. for 30 seconds and 60° C. for 90 seconds.

The relative telomerase activity is assessed against that of HEK cells using the threshold cycle number (or Ct value) for 1 μg protein cell lysate.

### Example 3

### Surface Antigen Analysis

Expression of cell surface antigens on fetal MSCs are analyzed using flow cytometry.

The cells are tryspinized for 5 minutes, centrifuged, resuspended in culture media and incubated in a bacterial culture dish for 1 hour in a 37° C., 5% CO₂ incubator. The cells are then collected, centrifuged, washed in 2% FBS. 2.5×10⁵ cells are then incubated with each of the following conjugated monoclonal antibodies: CD29-PE, CD44-FITC, CD49a-PE, CD49e-PE, CD105-FITC, CD166-PE, CD73-FITC, CD34-FITC, CD45-FITC (PharMingen, San Diego, Calif.) for 60 min on ice.

After incubation, cells are washed and resuspended in 2% FBS. Nonspecific fluorescence is determined by incubation of similar cell aliquots with isotype-matched mouse monoclonal antibodies (PharMingen, San Diego, Calif.). Data are analyzed by collecting 20,000 events on a BD FACSCalibur™ Flow Cytometer (BD Bioscences, San Jose, Calif.) instrument using CELLQuest software.

### Example 4

### Quantitative RT-PCR

Total RNA is extracted from cells using TRIzol® LS Reagent (Invitrogen Corporation, Carlsbad, Calif.) according to manufacturer's instruction.

Total RNA is converted to cDNA using the High Capacity cDNA Reverse Transcription Kit (Applied Biosystems Inc., Foster City, Calif.) that is based on oligo-dT primed reverse transcription. Real time PCR is performed on a StepOne™ Plus Real-Time PCR System (Applied Biosystems Inc, Foster City, Calif.) using 2× Fast SYBR® Green Master Mix (Applied Biosystems Inc, Foster City, Calif.) according to manufacturer's instruction. The primers for OCT4 are 5′-AGTGAACAGGGAATGGGTGAA-3′ and 5′-AAG CGG CAG ATG GTC GT-3′, and for SOX2 are 5′-TGAGAGAAAGAAGAGGAGAGA-3′ and 5′-TGGGGGAAAAAAAGAGAGAGG-3′.

### Example 5

### Illumina Gene Chip Analysis

Total RNA is prepared in technical triplicates from different passages of F1lb (p10, p12, p14), F1ki (p12, p14, p16), F2lb (p10, p12, p14), F3lb (p10, p12, p14) and F3li (p10, p16), and from two technical replicates of the previously described hESC-MSCs line, Hues9.E1 (p19).

500 ηg RNA is converted to biotinylated cRNA using the Illumina RNA Amplification Kit (Ambion, Inc., Austin, Tex.) according to the manufacturer's directions. 750 ng of the biotinylated cRNA are hybridized to the Sentrix HumanRef-8 Expression BeadChip Version 3 (Illumina, Inc., San Diego, Calif.), washing and scanning are performed according to the Illumina BeadStation 500× manual. The data are analyzed using Genespring GX 10. Quantile normalization is performed by a shift to 75^{th} percentile, and the normalized data are baseline transformed to the median of all samples.

### Example 6

### SDS-PAGE Analysis and Western Blot Hybridization

For SDS-PAGE analysis, total proteins in CM are separated on 4-12% SDS-polyacrylamide gels and stained with silver.

For western blot hybridization, after separation on a SDS-PAGE, the proteins are electroblotted onto a nitrocellulose membrane. The membrane is blocked, incubated with the primary anti-human antibodies that included 1:60 dilution of mouse anti-CD9, 1:60 dilution of mouse anti-CD81, 1:56 dilution of mouse anti-Alix, 1:200 dilution of mouse anti-pyruvate kinase (PK), 1:60 dilution of mouse anti-SOD-1 or 1:60 dilution of goat anti-TSP-1. The blot is then incubated with a horseradish peroxidase-coupled secondary antibody. The secondary antibodies used are 1:1250 or 1:1364 dilution of goat anti-mouse IgG or 1:1364 dilution of donkey anti-goat IgG.

All antibodies are obtained from Santa Cruz Biotechnology, Santa Cruz, Calif. except mouse anti-PK which is from Abeam Inc., Cambridge, Mass. The blot is then incubated with HRP-enhanced chemiluminescent substrate (Thermo Fisher Scientific Inc., Waltham, Mass.) and then exposed to a X-ray film.

### Example 7

### HPLC Purification of Microparticles

The instrument setup consisted of a liquid chromatography system with a binary pump, an auto injector, a thermostated column oven and a UV-visible detector operated by the Class VP software from Shimadzu Corporation (Kyoto, Japan).

The Chromatography columns used are TSK Guard column SWXL, 6×40 mm and TSK gel G4000 SWXL, 7.8×300 mm from Tosoh Corporation (Tokyo, Japan). The following detectors, Dawn 8 (light scattering), Optilab (refractive index) and QELS (dynamic light scattering) are connected in series following the UV-visible detector. The last three detectors are from Wyatt Technology Corporation (California, USA) and are operated by the ASTRA software.

The components of the sample are separated by size exclusion i.e. the larger molecules will elute before the smaller molecules. The eluent buffer used is 20 mM phosphate buffer with 150 mM of NaCl at pH 7.2. This buffer is filtered through a pore size of 0.1 μm and degassed for 15 minutes before use. The chromatography system is equilibrated at a flow rate of 0.5 ml/min until the signal in Dawn 8 stabilized at around 0.3 detector voltage units. The UV-visible detector is set at 220 ηm and the column is oven equilibrated to 25° C. The elution mode is isocratic and the run time is 40 minutes.

The volume of sample injected ranged from 50 to 100 The hydrodynamic radius, Rh is computed by the QELS and Dawn 8 detectors. The highest count rate (Hz) at the peak apex is taken as the Rh. Peaks of the separated components visualized at 220 ηm are collected as fractions for further characterization studies.

### Example 8

### Testing Secretions and Microparticles for Cardioprotection

The secretion is prepared by growing the fetal MSCs in a chemically defined serum free culture medium for three days as described²⁵.

This CM is collected, clarified by centrifugation, concentrated 50 times using tangential flow filtration with MW cut-off of 100 kDa (Satorius, Goettingen, Germany) and sterilized by filtration through a 220 nm filter. The CM is tested in a mouse model of ischemia and reperfusion injury. MI is induced by 30 minutes left coronary artery (LCA) occlusion and subsequent reperfusion. Five minutes before reperfusion, mice are intravenously infused with 200 μl saline diluted CM containing 3 μg protein for Hues9.E1 (hESC-MSCs) CM or 150 μg protein for fetal MSCs CM or 10 μg protein for HPLC F1 via the tail vein.

Control animals are infused with 200 μl saline. After 24 hours reperfusion, infarct size (IS) as a percentage of the area at risk (AAR) is assessed using Evans' blue dye injection and TTC staining as described previously²⁶.

### Example 9

### Statistical Analysis

Two-way ANOVA with post-hoc Dunnett is used to test the difference in infarct size between groups. Correlation coefficient of each pairs of array is assessed using Pearson correlation test.

### Example 10

### Generating MSCs Cultures from Human Fetal Tissues

We generated five MSC cultures from fetal limb (F1lb, F2lb, F3lb), kidney (F1ki) and liver (F3li) tissues of three fetuses in three independent experiments using feeder- and serum-free culture condition as described above and in²⁴.

Briefly, the cultures are cultured in either serum-free or serum-containing culture medium. When confluent, the cultures are passaged by trypsinizing and then splitting at 1:4 on a gelatinized tissue culture plate. All medium components are obtained from Invitrogen. Serum-free culture medium is made up of Knockout DMEM medium supplemented with 10% serum replacement media, non-essential amino acids, 10 ng/ml FGF2, 10 ng/ml Recombinant Human EGF and 55 μM β-mercaptoethanol. Serum-containing culture medium is made up of DMEM-high glucose without glutamine supplemented with penicillin-streptomycin, L-glutamine, non-essential amino acids and 10% fetal calf serum.

A homogenous culture of putative fibroblast-like MSCs migrated out of the tissues and adhered to the plastic culture dish, two days after fetal tissues are plated on gelatinized tissue culture plates. This observation is consistent with the defining characteristic of MSCs i.e. adherence to plastic. The large tissue pieces are then washed off leaving a homogenous cell culture. This procedure is performed on five different fetal tissues originating from three fetuses in three independent experiments.

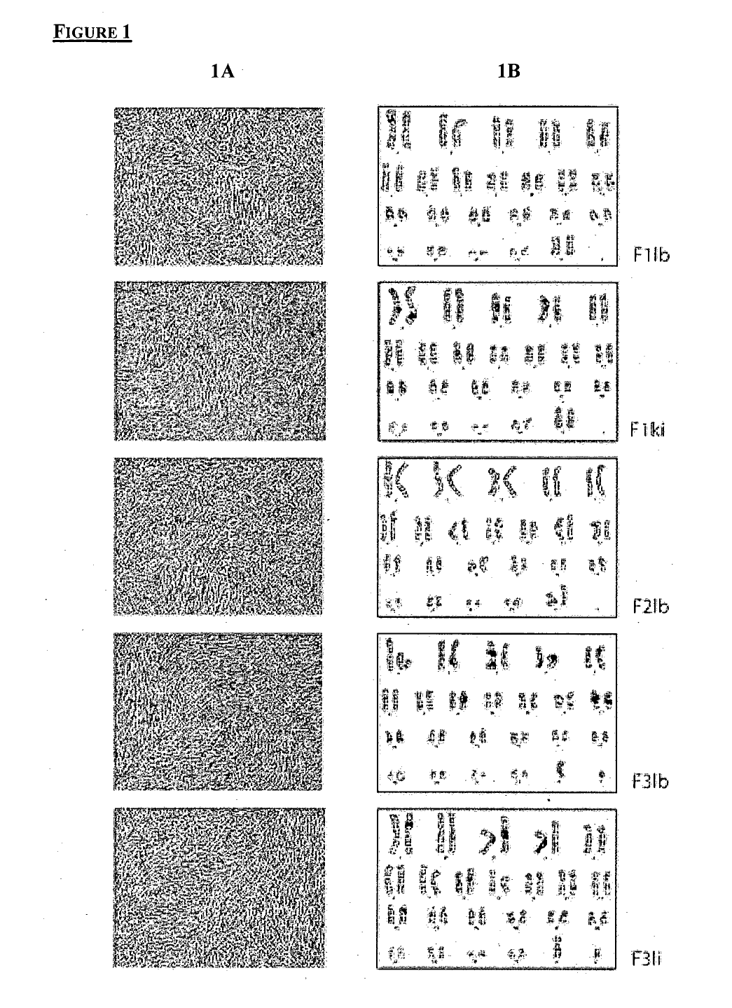

As shown in FIG. 1A, each time, a homogenous culture is obtained of putative MSCs that form a typical fingerprint whorl at confluency.

The cultures are designated P1 when 2×10⁷ cells are generated. The average population doubling time of all five cultures are between 72 to 96 hours and is most optimal at between 25-80% confluency or 15-50,000 cells per cm². The cells could be maintained in continuous culture for at least 20 passages at a 1:4 split. The karyotype of all five cultures at p10-12 is normal i.e. 46 XX or 46XY as determined by G-banding, as shown in FIG. 1B.

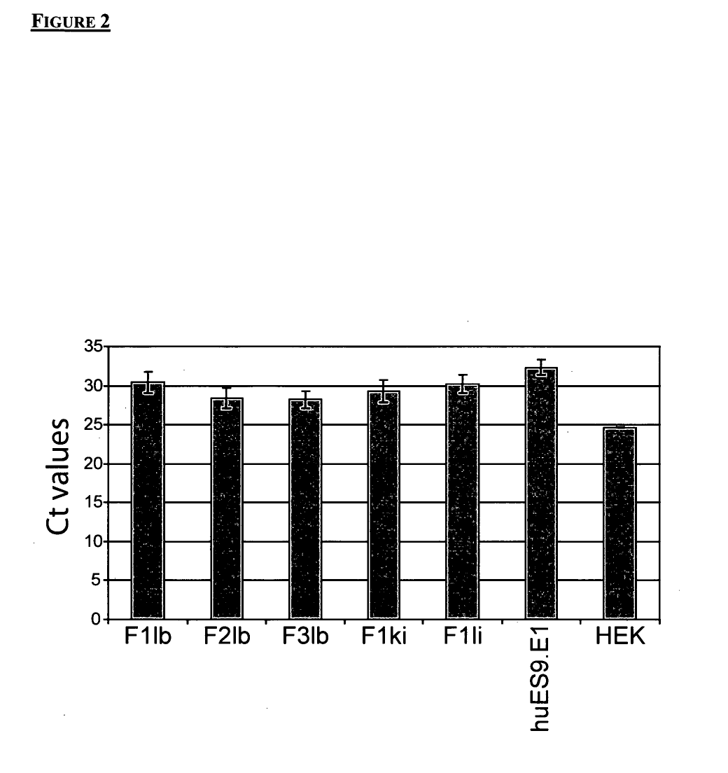

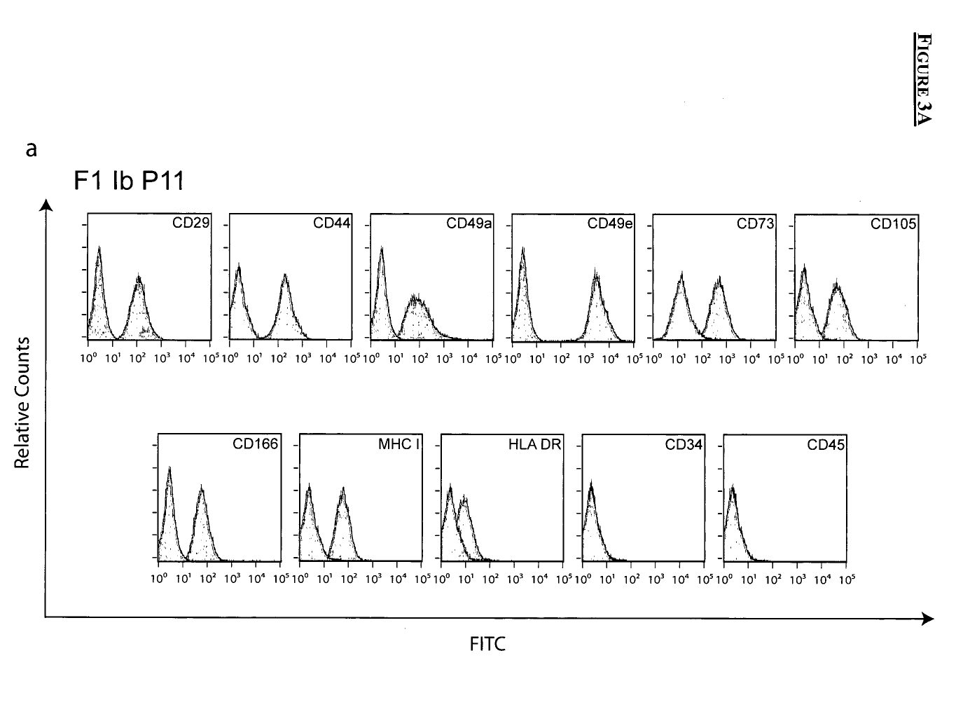

At passages 14, 16 and 18, telomerase activity in all five MSC cultures is determined and the average cellular telomerase activity over three passages in each of the five cultures is equivalent to that of the previously described Hues9.E1 hESC-MSCs²⁴. This is shown in FIG. 2.

### Example 11

### Assessment of Fetal Cultures as MSCs

The five putative MSC cultures derived from fetal tissues are assessed according to the ISCT minimal criteria for the definition of human MSCs²⁹.

All five presumptive MSC cultures derived from three different fetuses are grown on plastic culture dishes as a monolayer of adherent spindle-shaped cells, as shown in FIG. 1A.

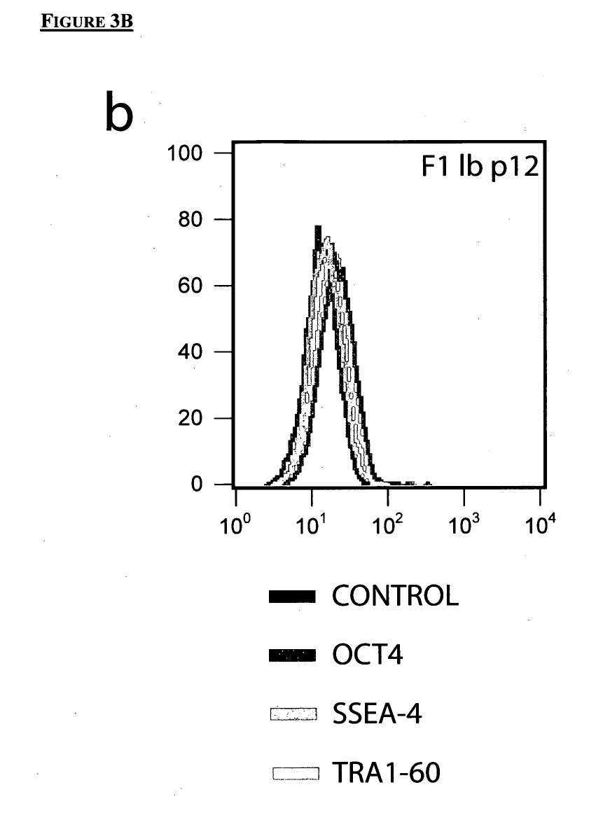

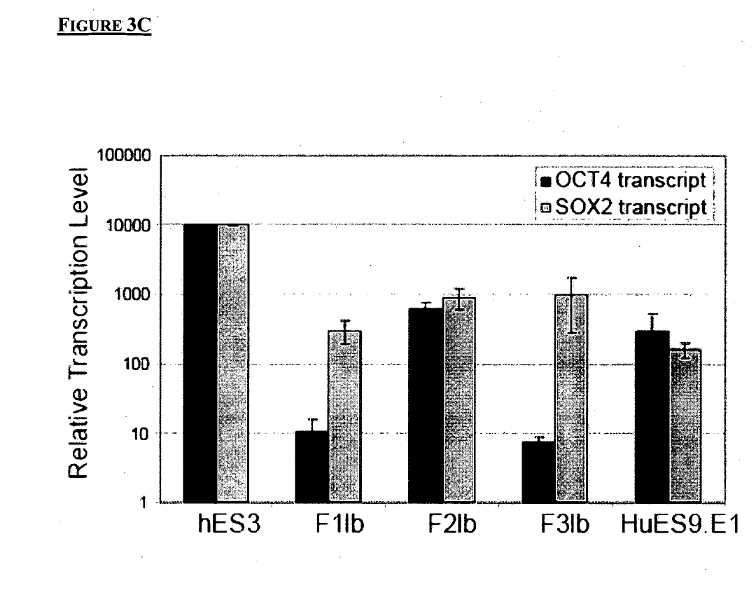

FIG. 3A shows that all five presumptive MSC cultures derived from three different fetuses are all CD29⁺, CD44⁺, CD49a⁺ CD49e⁺, CD105⁺, CD166⁺, MHC I⁺, CD34⁻ and CD45⁻ as represented by F1lb MSCs.

We observed that F1lb MSCs are HLA-DR^{lo} but the remaining four cultures are HLA-DR⁻. In contrast to previous reports³⁰⁻⁴², these fetal MSCs like hESC-MSCs did not express pluripotency-associated proteins Oct4, SSEA-4, Tra1-60 as exemplified by F1lb MSCs. This is shown in FIG. 3B

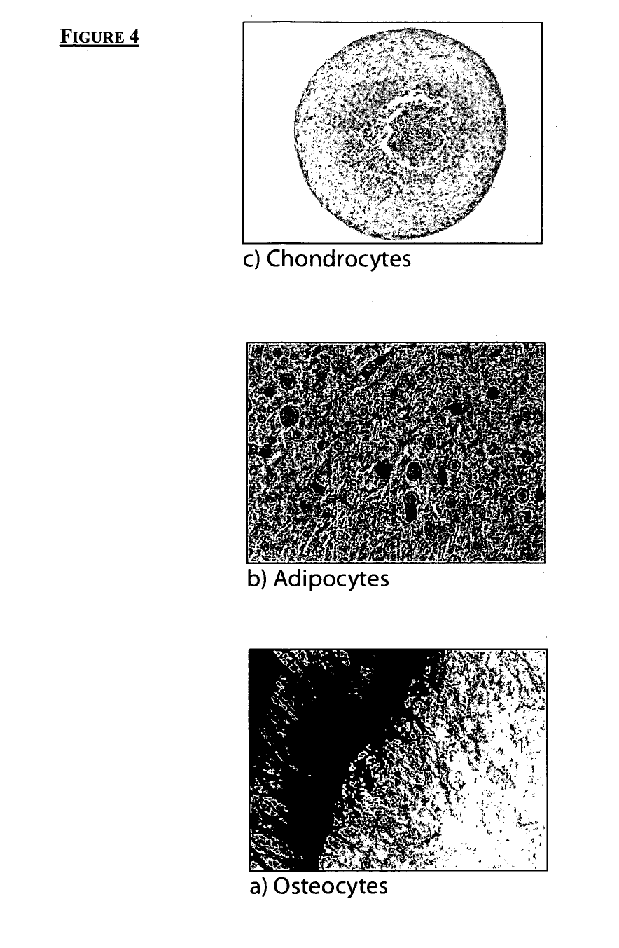

However, transcripts of OCT4 and SOX2 are readily detected by real time PCR but their levels are at least ten times lower than those in hES3 human ESCs (FIG. 3C). All five presumptive fetal MSC cultures could be induced to differentiate to osteoblasts, adipocytes and chondroblasts in vitro. This is shown in FIG. 4.

### Example 12

### Gene Expression Profile

Genome-wide gene expression profiling of the fetal MSCs and hESC-MSCs are performed using microarray hybridisation to assess (1) the relatedness among the five fetal MSC cultures derived from three different tissues with hESC-MSCs; (2) the relatedness between the fetal MSC cultures and hESC-MSCs.

Microarray hybridization are performed in duplicate on Sentrix Human Ref-8 Expression BeadChip version 3 (Illumina, Inc., San Diego, Calif.) using RNA from two or three different passages of the MSC cultures.

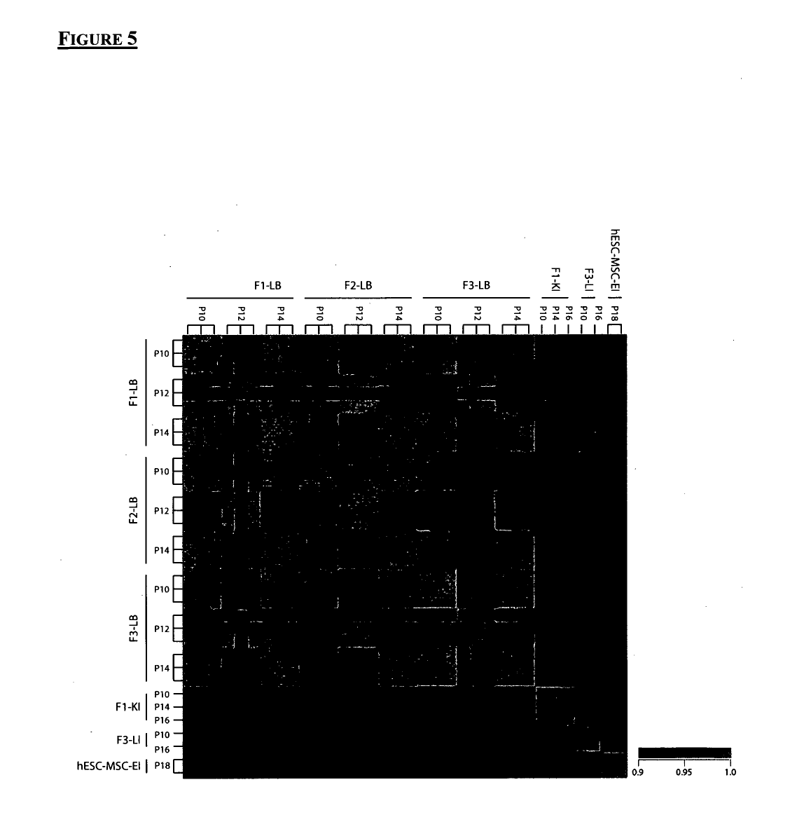

The gene expression profile between different passages of each culture, between the five cultures or between each fetal MSCs and hESC-MSCs is highly similar with a correlation value of >0.9. This is shown in FIG. 5.

### Example 13

### Cardioprotective Activity of Secretion

Secretion by two of the fetal MSC cultures, F1lb and F1ki are tested for cardioprotective activity in a mouse model of myocardial ischemia and reperfusion injury as previously described²⁶.

Briefly, the cultures are grown in a chemically defined medium and the secretion harvested as we have previously described²⁵.

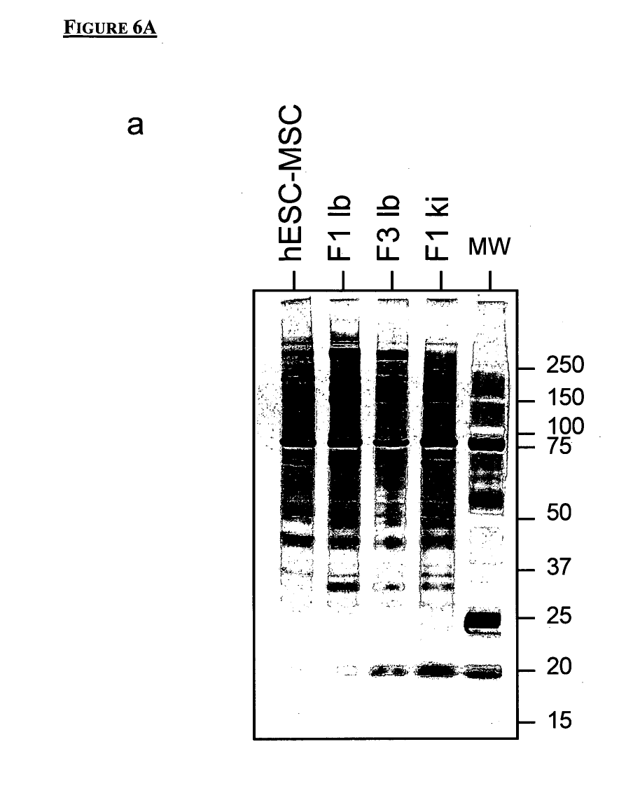

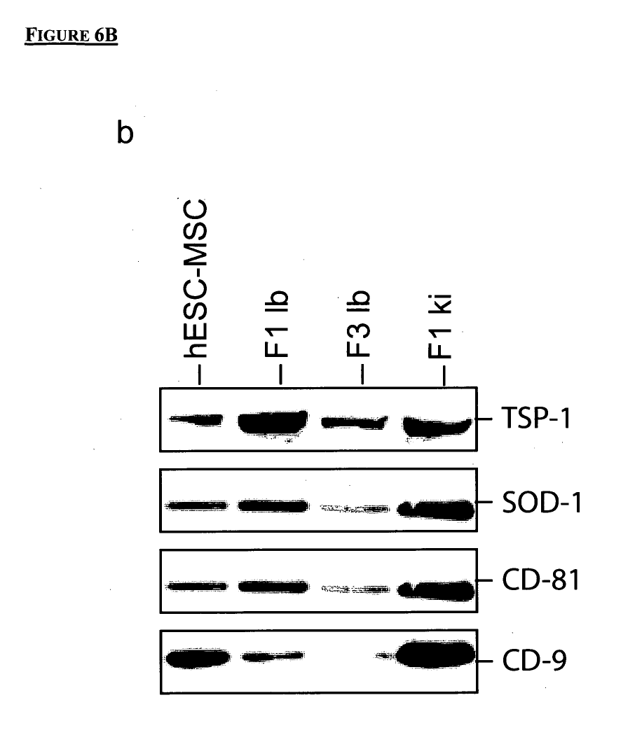

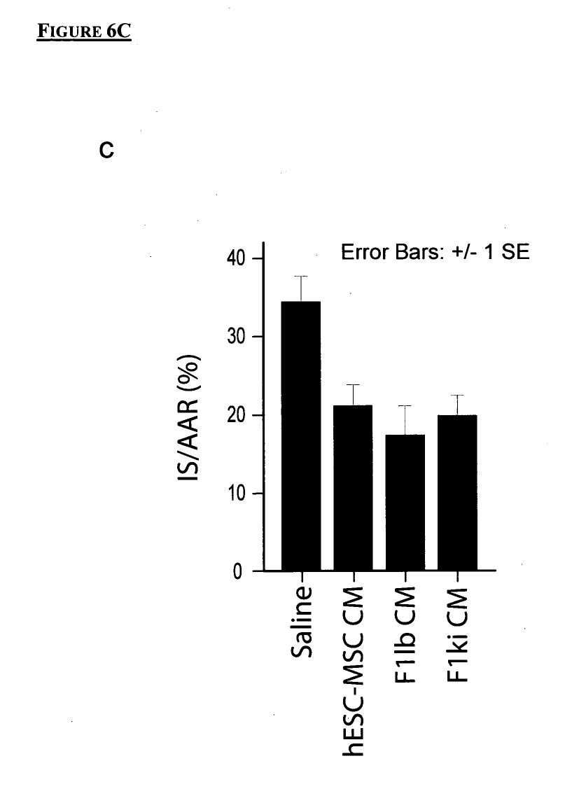

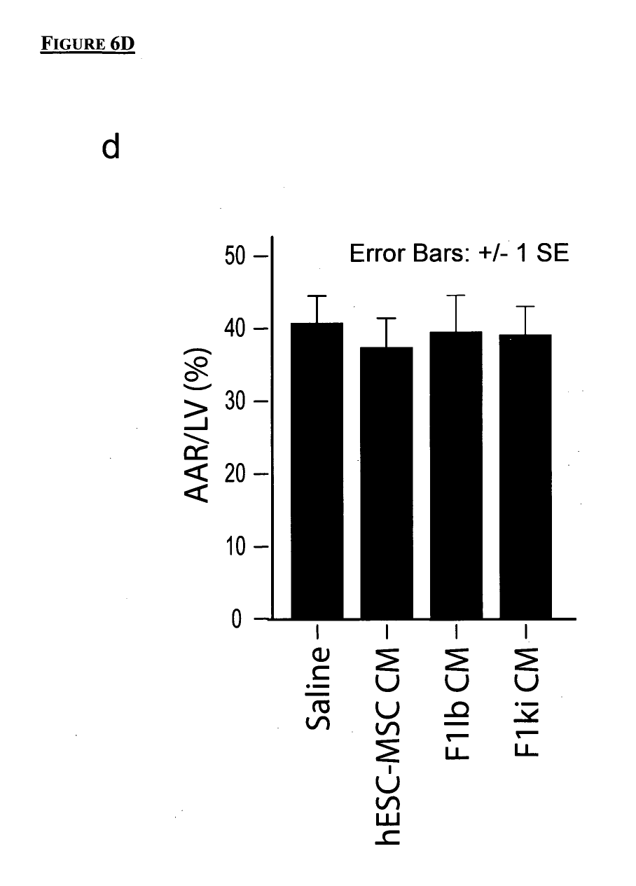

The gross protein composition of secretion from both F1lb and F1ki as determined by silver staining of proteins resolved on a one-D SDS-PAGE appear similar to each other, to that of Hues9.E1 hESC-MSCs and also to that from F3lb derived from fetal limb tissues of a different sex. This is shown in FIG. 6A.

However, the relative abundance of specific proteins such as TSP-1, SOD-1, CD81 and CD9 are different among all the secretions, as shown in FIG. 6B.

Nevertheless, the secretion from either F1lb or F1ki when administered to a mouse model of myocardial ischemia-reperfusion injury significantly reduced infarct size to the same extent as mice treated with hESC-MSCs CM. Conditioned medium from F1lb, F1ki and hESC-MSCs reduced infarct size (IS) with 50%, 42% and 39% respectively (p<0.05). This is shown in FIG. 6C.

The area at risk (AAR) as a percentage of the left ventricular (LV) wall is similar in all the mice tested, as shown in FIG. 6D.

### Example 15

### Microparticles Mediate the Cardioprotective Effects of the Secretion

We had previously shown that the cardioprotection effects of secretion from hESC-MSCs are mediated by large complexes of ˜1000 kDa²⁶.

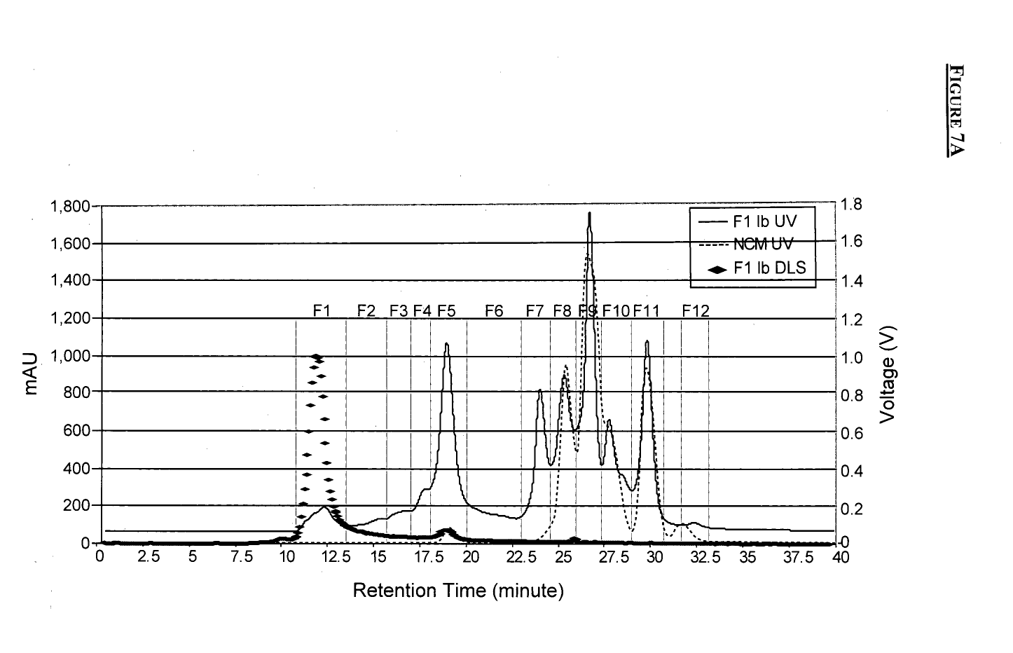

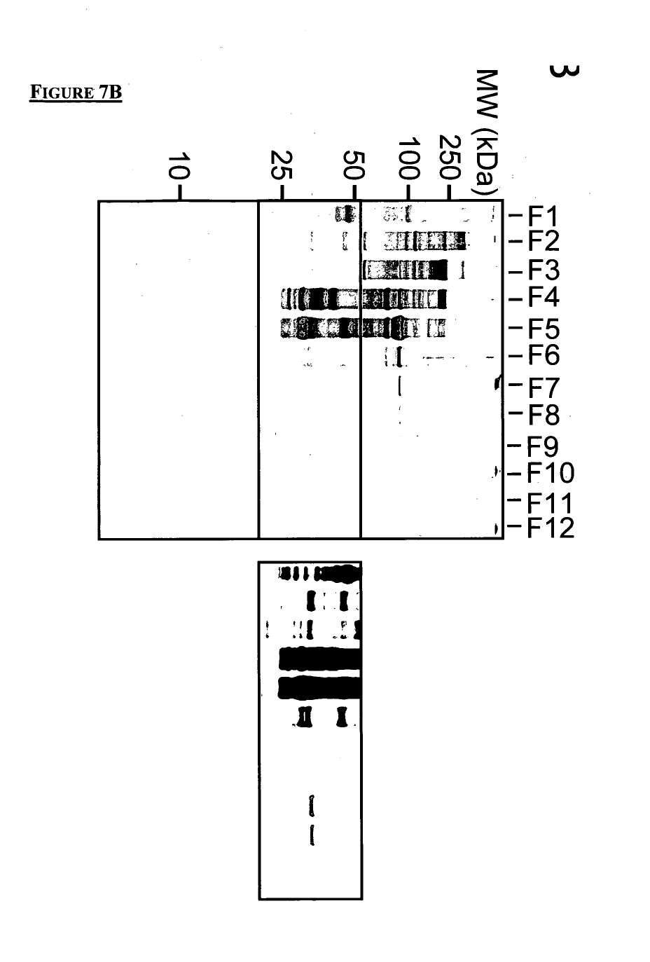

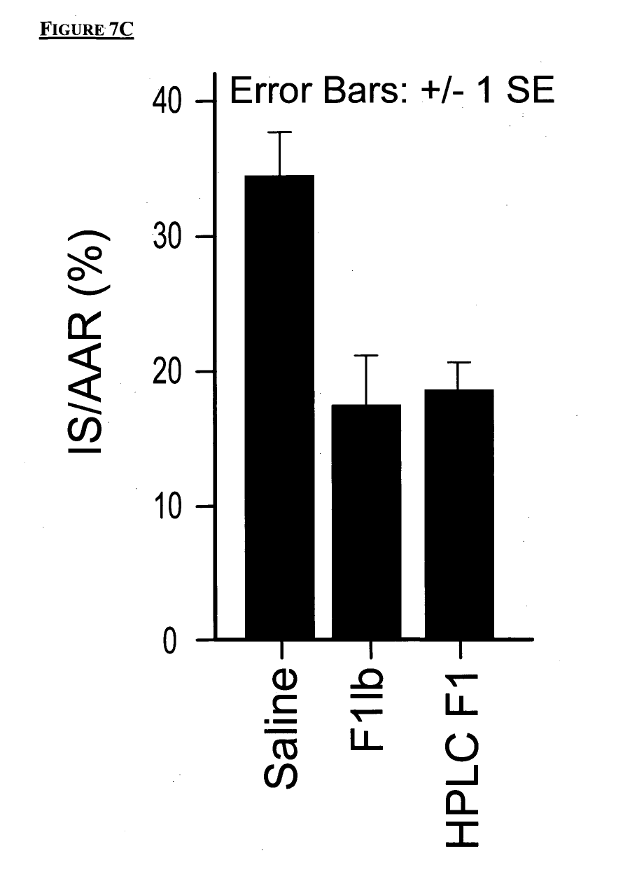

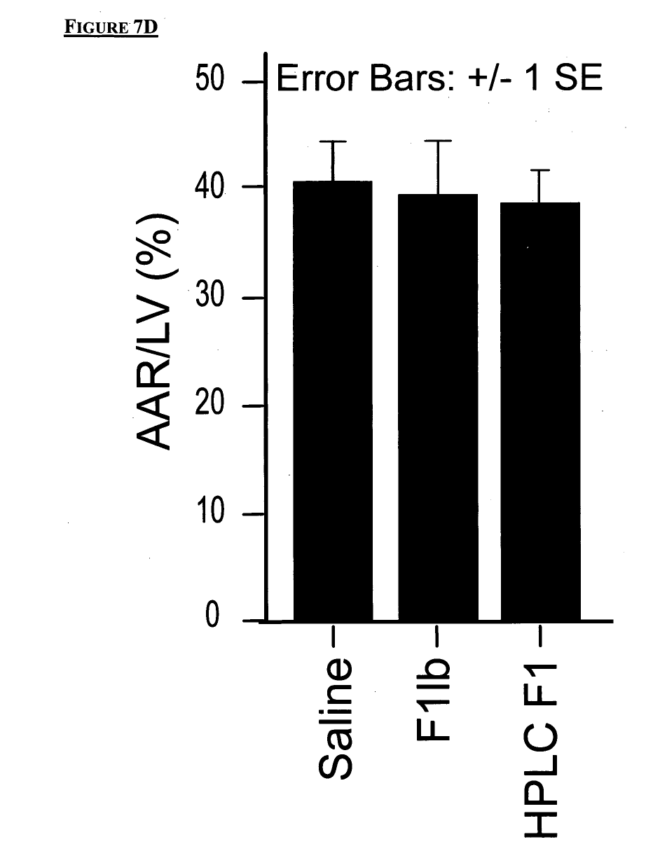

To determine if there are such complexes in the secretion of the fetal MSCs, the CM is fractionated by size exclusion on a HPLC column. We observed five fractions that are present in the CM but not the NCM (FIG. 7A).

These five fractions, F1-F5 therefore represent secretions from the MSCs. In a size exclusion fractionation where larger molecules are eluted faster than smaller ones, we observe that only proteins in fraction F2 to F5 followed this principle of fractionation. Proteins in the fastest eluting F1 fraction contained proteins that spanned in entire MW spectrum of F2 to F5. This suggested that the proteins in F1 are in large aggregates (FIG. 7B).

To confirm this, sizes of molecules in the five fractions are determined by dynamic light scattering analysis which has detection range of 1 to 1000 ηm. The sizes of molecules in fractions F2 to F5 are too heterogenous to be determined by dynamic light scattering analysis. In spite of the wide MW spectrum of proteins in F1, the size of molecules in F1 fraction is sufficiently homogenous to be determined as having a hydrodynamic radius of 50-65 ηm by dynamic light scattering analysis.

When administered to the mouse model of mouse model of myocardial ischemia-reperfusion injury as described above, these HPLC-purified microparticles reduced infarct size at < 1/10 dosage of the secretion (FIG. 7C and FIG. 7D).

### Example 14

### Discussion

The trophic effects of MSCs transplantation on ameliorating the deleterious consequences of myocardial ischemia have been implicated in several studies¹¹.

Transplantation of MSCs into ischemic myocardium has been shown to induce several tissue responses such as an increased production of angiogenic factors and decreased apoptosis⁴³ that were better explained by secretion of paracrine factors than by differentiation of MSCs, the so-called paracrine hypothesis.

Gnecchi et al demonstrated that culture medium conditioned by Akt-transformed BM-MSCs was as effective as transplantation of the cells themselves¹⁰ but that secretion from untransformed rat BM-MSCs was not as cardioprotective as that from Akt-transformed BM-MSCs¹⁷ suggesting that the difference in the biological activity of secretion from hESC-MSCs and rodent adult BM-MSCs may be due to the developmental stage of the MSCs i.e. embryonic versus adult.

To determine that the cardioprotective secretion of hESC-MSCs is a bona fide property of non-adult human MSCs, we derived human MSCs directly from fetal human tissues. Like hESC-MSCs, these fetal MSCs fulfilled the basic criteria for MSCs as defined by The International Society for Cellular Therapy²⁹. They adhered to plastic, have a typical MSCs-like surface antigen profile as defined by the presence of surface antigens such as CD29, CD44, CD49a, CD49e, CD105, CD166, MHC I and the absence of surface antigens such as HLA-DR, CD34 and CD45⁴⁴⁻⁴⁶, and a typical MSCs differentiation potential that includes adipogenesis, chondrogenesis and osteogenesis.

Irrespective of their tissue of origin, the five fetal MSC cultures derived from different tissues of three individual fetuses have a nearly identical genome-wide gene expression profile. Their gene expression profiles were also similar to that of the previously described hESC-MSCs, Hues9.E1. They were as equally proliferative as Hues9.E1 and had high levels of telomerase activity.

Most importantly, the secretion by MSCs derived from different tissues i.e. limb and kidney tissues significantly reduced infarct size in a mouse model of ischemia/reperfusion injury, suggesting that this is typical for non-adult MSCs.

In conclusion, we show that both embryonic stem cell and fetal tissue derived MSCs produce cardioprotective secretion, and that these fetal cells are an alternative cell source for secretion that can reduce infarct size after ischemia/reperfusion injury.

Consistent with our previous observation that the cardioprotective effect of secretion from hESC-MSCs was mediated by large >1000 kDa complexes²⁶, we demonstrate that the cardioprotective effects of secretion from fetal MSCs are also mediated by large particles with a hydrodynamic radius of 55-85 ηm.

Although microparticles are known to be produced by numerous cell types and have been implicated in many diseases⁴⁷⁻⁵⁶, this is the first report that MSC produced microparticles with a cardioprotective effect. This involvement of microparticles in mediating the paracrine effect of MSC transplantation on tissue repair represents a radical shift in our current understanding of the paracrine effect of MSC which hitherto has been limited to cytokine, chemokine or growth factor-mediated extracellular signaling¹¹. The relatively simple purification of these microparticles from a highly scalable cell source also engenders a viable strategy addressing an urgent unmet therapeutic need for treating myocardial ischemia-reperfusion injury⁵⁷.

### REFERENCES

1. Le Blanc K, Pittenger M. Mesenchymal stem cells: progress toward promise. Cytotherapy. 2005; 7:36-45.
2. Minguell J J, Erices A. Mesenchymal stem cells and the treatment of cardiac disease. Exp Biol Med (Maywood). 2006; 231:39-49.
3. Schuleri K H, Boyle A J, Hare J M. Mesenchymal stem cells for cardiac regenerative therapy. Handb Exp Pharmacol. 2007:195-218.
4. Wang X J, Li Q P. The roles of mesenchymal stem cells (MSCs) therapy in ischemic heart diseases. Biochem Biophys Res Commun. 2007; 359:189-193.
5. Abdel-Latif A, Bolli R, Tleyjeh I M, et al. Adult bone marrow-derived cells for cardiac repair: a systematic review and meta-analysis. Arch Intern Med. 2007; 167:989-997.
6. Mazhari R, Hare J M. Advances in cell-based therapy for structural heart disease. Prog Cardiovasc Dis. 2007; 49:387-395.
7. Ohnishi S, Nagaya N. Prepare cells to repair the heart: mesenchymal stem cells for the treatment of heart failure. Am J. Nephrol. 2007; 27:301-307.
8. Behfar A, Terzic A. Optimizing adult mesenchymal stem cells for heart repair. J Mol Cell Cardiol. 2007; 42:283-284.
9. Atsma D E, Fibbe W E, Rabelink T J. Opportunities and challenges for mesenchymal stem cell-mediated heart repair. Curr Opin Lipidol. 2007; 18:645-649.
10. Gnecchi M, He H, Liang O D, et al. Paracrine action accounts for marked protection of ischemic heart by Akt-modified mesenchymal stem cells. Nat. Med. 2005; 11:367-368.
11. Caplan A I, Dennis J E. Mesenchymal stem cells as trophic mediators. J Cell Biochem. 2006; 98:1076-1084.
12. Kinnaird T, Stabile E, Burnett M S, et al. Local delivery of marrow-derived stromal cells augments collateral perfusion through paracrine mechanisms. Circulation. 2004; 109:1543-1549.
13. Leedham S J, Brittan M, McDonald S A, et al. Intestinal stem cells. J Cell Mol Med. 2005; 9:11-24.
14. Togel F, Hu Z, Weiss K, et al. Administered mesenchymal stem cells protect against ischemic acute renal failure through differentiation-independent mechanisms. Am J Physiol Renal Physiol. 2005; 289:F31-42.
15. Patschan D, Plotkin M, Goligorsky M S. Therapeutic use of stem and endothelial progenitor cells in acute renal injury: ca ira. Curr Opin Pharmacol. 2006; 6:176-183.
16. Miyahara Y, Nagaya N, Kataoka M, et al. Monolayered mesenchymal stem cells repair scarred myocardium after myocardial infarction. Nat. Med. 2006; 12:459-465.
17. Gnecchi M, He H, Noiseux N, et al. Evidence supporting paracrine hypothesis for Akt-modified mesenchymal stem cell-mediated cardiac protection and functional improvement. Faseb J. 2006; 20:661-669.
18. Mayer H, Bertram H, Lindenmaier W, et al. Vascular endothelial growth factor (VEGF-A) expression in human mesenchymal stem cells: autocrine and paracrine role on osteoblastic and endothelial differentiation. J Cell Biochem. 2005; 95:827-839.
19. Nakagami H, Maeda K, Morishita R, et al. Novel autologous cell therapy in ischemic limb disease through growth factor secretion by cultured adipose tissue-derived stromal cells. Arterioscler Thromb Vasc Biol. 2005; 25:2542-2547.
20. Van Overstraeten-Schlogel N, Beguin Y, Gothot A. Role of stromal-derived factor-1 in the hematopoietic-supporting activity of human mesenchymal stem cells. Eur J Haematol. 2006; 76:488-493.
21. Cheng L, Qasba P, Vanguri P, et al. Human mesenchymal stem cells support megakaryocyte and pro-platelet formation from CD34(+) hematopoietic progenitor cells. J Cell Physiol. 2000; 184:58-69.
22. Mirotsou M, Zhang Z, Deb A, et al. Secreted frizzled related protein 2 (Sfrp2) is the key Akt-mesenchymal stem cell-released paracrine factor mediating myocardial survival and repair. Proc Natl Acad Sci USA. 2007; 104:1643-1648.
23. Dai W, Hale S L, Kloner R A. Role of a paracrine action of mesenchymal stem cells in the improvement of left ventricular function after coronary artery occlusion in rats. Regen Med. 2007; 2:63-68.
24. Lian Q, Lye E, Suan Yeo K, et al. Derivation of Clinically Compliant MSCs from CD105+, CD24− Differentiated Human ESCs. Stem Cells. 2007; 25:425-436.
25. Sze S K, de Kleijn D P, Lai R C, et al. Elucidating the secretion proteome of human embryonic stem cell-derived mesenchymal stem cells. Mol Cell Proteomics. 2007; 6:1680-1689.
26. Timmers L, Lim S-K, Arslan F, et al. Reduction of myocardial infarct size by human mesenchymal stem cell conditioned medium. Stem Cell Research. 2008; 1:129-137.
27. Ethical, Legal and Social Issues in Human Stem Cell Research, Reproductive and Therapeutic Cloning. Singapore Bioethics Advisory Committee Report. 2002.
28. Wege H, Chui M S, Le H T, et al. SYBR Green real-time telomeric repeat amplification protocol for the rapid quantification of telomerase activity. Nucleic Acids Res. 2003; 31:E3-3.
29. Dominici M, Le Blanc K, Mueller I, et al. Minimal criteria for defining multipotent mesenchymal stromal cells. The International Society for Cellular Therapy position statement. Cytotherapy. 2006; 8:315-317.
30. Tsai M S, Lee J L, Chang Y J, et al. Isolation of human multipotent mesenchymal stem cells from second-trimester amniotic fluid using a novel two-stage culture protocol. Hum Reprod. 2004; 19:1450-1456.
31. Bossolasco P, Montemurro T, Cova L, et al. Molecular and phenotypic characterization of human amniotic fluid cells and their differentiation potential. Cell Res. 2006; 16:329-336.
32. Fong C Y, Richards M, Manasi N, et al. Comparative growth behaviour and characterization of stem cells from human Wharton's jelly. Reprod Biomed Online. 2007; 15:708-718.
33. Guillot P V, Gotherstrom C, Chan J, et al. Human first-trimester fetal MSC express pluripotency markers and grow faster and have longer telomeres than adult MSC. Stem Cells. 2007; 25:646-654.
34. Kim J, Lee Y, Kim H, et al. Human amniotic fluid-derived stem cells have characteristics of multipotent stem cells. Cell Prolif. 2007; 40:75-90.
35. Peng H H, Wang T H, Chao A S, et al. Isolation and differentiation of human mesenchymal stem cells obtained from second trimester amniotic fluid; experiments at Chang Gung Memorial Hospital. Chang Gung Med J. 2007; 30:402-407.
36. Roubelakis M G, Pappa K I, Bitsika V, et al. Molecular and proteomic characterization of human mesenchymal stem cells derived from amniotic fluid: comparison to bone marrow mesenchymal stem cells. Stem Cells Dev. 2007; 16:931-952.
37. Yen M L, Chien C C, Chiu I M, et al. Multilineage differentiation and characterization of the human fetal osteoblastic 1.19 cell line: a possible in vitro model of human mesenchymal progenitors. Stem Cells. 2007; 25:125-131.
38. Jo C H, Kim O S, Park E Y, et al. Fetal mesenchymal stem cells derived from human umbilical cord sustain primitive characteristics during extensive expansion. Cell Tissue Res. 2008; 334:423-433.
39. Kermani A J, Fathi F, Mowla S J. Characterization and genetic manipulation of human umbilical cord vein mesenchymal stem cells: potential application in cell-based gene therapy. Rejuvenation Res. 2008; 11:379-386.
40. Poloni A, Rosini V, Mondini E, et al. Characterization and expansion of mesenchymal progenitor cells from first-trimester chorionic villi of human placenta. Cytotherapy. 2008; 10:690-697.
41. Wang X Y, Lan Y, He W Y, et al. Identification of mesenchymal stem cells in aorta-gonad-mesonephros and yolk sac of human embryos. Blood. 2008; 111:2436-2443.
42. Zhang Z Y, Teoh S H, Chong M S, et al. Superior Osteogenic Capacity for Bone Tissue Engineering of Fetal Compared To Perinatal and Adult Mesenchymal Stem Cells. Stem Cells. 2008.
43. Tang Y L, Zhao Q, Qin X, et al. Paracrine action enhances the effects of autologous mesenchymal stem cell transplantation on vascular regeneration in rat model of myocardial infarction. Ann Thorac Surg. 2005; 80:229-236; discussion 236-227.
44. Javazon E H, Beggs K J, Flake A W. Mesenchymal stem cells: paradoxes of passaging. Exp Hematol. 2004; 32:414-425.
45. Barry F P, Murphy J M. Mesenchymal stem cells: clinical applications and biological characterization. Int J Biochem Cell Biol. 2004; 36:568-584.
46. Majumdar M K, Keane-Moore M, Buyaner D, et al. Characterization and functionality of cell surface molecules on human mesenchymal stem cells. J Biomed Sci. 2003; 10:228-241.
47. Meziani F, Tesse A, Andriantsitohaina R. Microparticles are vectors of paradoxical information in vascular cells including the endothelium: role in health and diseases. Pharmacol Rep. 2008; 60:75-84.
48. Nomura S, Ishii K, Inami N, et al. Evaluation of angiopoietins and cell-derived microparticles after stem cell transplantation. Biol Blood Marrow Transplant. 2008; 14:766-774.
49. Redman C W, Sargent I L. Circulating microparticles in normal pregnancy and pre-eclampsia. Placenta. 2008; 29 Suppl A:S73-77.
50. Shet A S. Characterizing blood microparticles: technical aspects and challenges. Vasc Health Risk Manag. 2008; 4:769-774.
51. Zwicker J I. Tissue factor-bearing microparticles and cancer. Semin Thromb Hemost. 2008; 34:195-198.
52. Aharon A, Brenner B. Microparticles, thrombosis and cancer. Best Pract Res Clin Haematol. 2009; 22:61-69.
53. Alijotas-Reig J, Palacio-Garcia C, Vilardell-Tarres M. Circulating microparticles, lupus anticoagulant and recurrent miscarriages. Eur J Obstet Gynecol Reprod Biol. 2009; 145:22-26.
54. Burnier L, Fontana P, Kwak B R, Angelillo-Scherrer A. Cell-derived microparticles in haemostasis and vascular medicine. Thromb Haemost. 2009; 101:439-451.
55. Castellana D, Kunzelmann C, Freyssinet J M. Pathophysiologic significance of procoagulant microvesicles in cancer disease and progression. Hamostaseologie. 2009; 29:51-57.
56. Chironi G N, Boulanger C M, Simon A, Dignat-George F, Freyssinet J M, Tedgui A. Endothelial microparticles in diseases. Cell Tissue Res. 2009; 335:143-151.
57. Knight D R. Editorial overview: cardioprotective drugs for myocardial ischemic injury—a therapeutic area at risk. Curr Opin Investig Drugs. 2007; 8:190-192.
A72. Spergel J M, Mizoguchi E, Brewer J P, Martin T R, Bhan A K, et al. (1998) Epicutaneous sensitization with protein antigen induces localized allergic dermatitis and hyperresponsiveness to methacholine after single exposure to aerosolized antigen in mice. J Clin Invest 101: 1614-1622.
A73. Huang C H, Kuo I C, Xu H, Lee Y S, Chua K Y (2003) Mite allergen induces allergic dermatitis with concomitant neurogenic inflammation in mouse. J Invest Dermatol 121: 289-293.
A74. Fernandes D J, Bonacci J V, Stewart A G (2006) Extracellular matrix, integrins, and mesenchymal cell function in the airways. Curr Drug Targets 7: 567-577.
A75. Camateros P, Moisan J, Henault J, De Sanctis J, Skamene E, et al. (2006) Toll-like receptors, cytokines and the immunotherapeutics of asthma. Curr Pharm Des 12: 2365-2374.
A76. Boxall C, Holgate S T, Davies D E (2006) The contribution of transforming growth factor-beta and epidermal growth factor signalling to airway remodelling in chronic asthma. Eur Respir J 27: 208-229.
A77. Howell J E, McAnulty R J (2006) TGF-beta: its role in asthma and therapeutic potential. Curr Drug Targets 7: 547-565.
A78. Scheller J, Ohnesorge N, Rose-John S (2006) Interleukin-6 trans-signalling in chronic inflammation and cancer. Scand J Immunol 63: 321-329.
A79. Peebles R S, Jr., Dworski R, Collins R D, Jarzecka K, Mitchell D B, et al. (2000) Cyclooxygenase inhibition increases interleukin 5 and interleukin 13 production and airway hyperresponsiveness in allergic mice. Am J Respir Crit Care Med 162: 676-681.
A80. Smaldone G C (2006) Advances in aerosols: adult respiratory disease. J Aerosol Med 19: 36-46.
A81. Lange C F, Finlay W H (2006) Liquid atomizing: nebulizing and other methods of producing aerosols. J Aerosol Med 19: 28-35.
A82. Devadason S G (2006) Recent advances in aerosol therapy for children with asthma. J Aerosol Med 19: 61-66.
A83. Groneberg D A, Paul H, Welte T (2006) Novel strategies of aerosolic pharmacotherapy. Exp Toxicol Pathol 57 Suppl 2: 49-53.
A84. Janssens H M, Tiddens H A (2006) Aerosol therapy: the special needs of young children. Paediatr Respir Rev 7 Suppl 1: S83-85.
A85. Scheuch G, Kohlhaeufl M J, Brand P, Siekmeier R (2006) Clinical perspectives on pulmonary systemic and macromolecular delivery. Adv Drug Deliv Rev 58: 996-1008.
A86. Acerbi D, Brambilla G, Kottakis I (2006) Advances in asthma and COPD management: Delivering CFC-free inhaled therapy using Modulite® technology. Pulm Pharmacol Ther.
A87. Ryan G, Pandit A, Apatsidis D P (2006) Fabrication methods of porous metals for use in orthopaedic applications. Biomaterials 27: 2651-2670.
A88. Oreffo R O, Triffitt J T (1999) Future potentials for using osteogenic stem cells and biomaterials in orthopedics. Bone 25: 5S-9S.
A89. Oakes B W (2004) Orthopaedic tissue engineering: from laboratory to the clinic. Med J Aust 180: S35-38.
A90. LeBaron R G, Athanasiou K A (2000) Extracellular matrix cell adhesion peptides: functional applications in orthopedic materials. Tissue Eng 6: 85-103.
A91. Geiger F, Bertram H, Berger I, Lorenz H, Wall O, et al. (2005) Vascular endothelial growth factor gene-activated matrix (VEGF165-GAM) enhances osteogenesis and angiogenesis in large segmental bone defects. J Bone Miner Res 20: 2028-2035.
A92. Mow V C, Ratcliffe A, Rosenwasser M P, Buckwalter J A (1991) Experimental studies on repair of large osteochondral defects at a high weight bearing area of the knee joint: a tissue engineering study. J Biomech Eng 113: 198-207.
A93. Willers C, Chen J, Wood D, Xu J, Zheng M H (2005) Autologous chondrocyte implantation with collagen bioscaffold for the treatment of osteochondral defects in rabbits. Tissue Eng 11: 1065-1076.
A94. Morley S J, Pain V M (1995) Hormone-induced meiotic maturation in Xenopus oocytes occurs independently of p70s6k activation and is associated with enhanced initiation factor (eIF)-4F phosphorylation and complex formation. J Cell Sci 108 (Pt 4): 1751-1760.
A95. Devine S M, Hoffman R (2000) Role of mesenchymal stem cells in hematopoietic stem cell transplantation. Curr Opin Hematol 7: 358-363.
A96. Koc O N, Gerson S L, Cooper B W, Dyhouse S M, Haynesworth S E, et al. (2000) Rapid hematopoietic recovery after coinfusion of autologous-blood stem cells and culture-expanded marrow mesenchymal stem cells in advanced breast cancer patients receiving high-dose chemotherapy. J Clin Oncol 18: 307-316.
A97. Maitra B, Szekely E, Gjini K, Laughlin M J, Dennis J, et al. (2004) Human mesenchymal stem cells support unrelated donor hematopoietic stem cells and suppress T-cell activation. Bone Marrow Transplant 33: 597-604.
A98. Aggarwal S, Pittenger M F (2005) Human mesenchymal stem cells modulate allogeneic immune cell responses. Blood 105: 1815-1822.
A99. Miura Y, Gao Z, Miura M, Seo B M, Sonoyama W, et al. (2006) Mesenchymal stem cell-organized bone marrow elements: an alternative hematopoietic progenitor resource. Stem Cells 24: 2428-2436.
A100. Augello A, Tasso R, Negrini S M, Amateis A, Indiveri F, et al. (2005) Bone marrow mesenchymal progenitor cells inhibit lymphocyte proliferation by activation of the programmed death 1 pathway. Eur J Immunol 35: 1482-1490.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1-15. (canceled)

16. A method for the treatment of or protection from effects of a disease or disorder, the method comprising administering to an individual in need thereof a conditioned medium preparation comprising medium conditioned by a fetal mesenchymal stem cell.

17. The method of claim 16, wherein the conditioned medium preparation comprises cardioprotective activity as assayed in an animal model of acute myocardial infarction.

18. The method of claim 16, wherein the conditioned medium preparation comprises a particle secreted by a fetal mesenchymal stem cell.

19. The method of claim 18, wherein said particle comprises an exosome.

20. The method of claim 19, wherein said exosome comprises at least cardioprotective activity as assayed in an animal model of acute myocardial infarction.

21. The method of claim 16, wherein said mesenchymal stem cell was obtained by a method comprising:
(a) providing an isolated fetal tissue;
(b) contacting the fetal tissue with a plastic surface;
(c) allowing fetal mesenchymal stem cells of said tissue to adhere to the plastic surface; and
(d) culturing said mesenchymal stem cells in serum-feee medium supplemented with 10% serum-replacement medium, non-essential amino acids, 10 ng/ml FGF2, 10 ng/ml recombinant human EGF and 55 μM β-mercaptoethanol.

22. The method of claim 16, wherein the fetal mesenchymal stem cell is a human cell.

23. The method of claim 16, wherein the disease or disorder is selected from the group consisting of cardiac diseases or disorders, reperfusion injury, myocardial infarction, a wound or scar, orthopedic disease, bone marrow disease, skin disease, burns and degenerative diseases.

24. The method of claim 16, wherein the conditioned medium preparation provides treatment of or protection against cardiac ischemic or reperfusion injury.

25. A method for the treatment of or protection from effects of a disease or disorder comprising administering to an individual in need thereof a preparation comprising an exosome isolated from medium conditioned by a fetal mesenchymal stem cell.

26. The method of claim 25, wherein the exosome is isolated from medium conditioned by a human cell.

27. The method of claim 25, wherein the exosome comprises at least cardioprotective activity as assayed in an animal model of acute myocardial infarction.

28. The method of claim 25, wherein the disease or disorder is selected from the group consisting of cardiac diseases or disorders, reperfusion injury, myocardial infarction, a wound or scar, orthopedic disease, bone marrow disease, skin disease, burns and degenerative diseases.

29. The method of claim 25, wherein the preparation comprising an exosome provides treatment of or protection against cardiac ischemic or reperfusion injury.

30. A method of producing an exosome, the method comprising culturing a fetal mesenchymal stem cell, cell culture or cell line in a cell culture medium and isolating the exosome from the conditioned cell culture medium.

31. The method of claim 30, wherein the mesenchymal stem cell, cell culture or cell line is a human cell.

32. An exosome composition isolated from medium conditioned by a fetal mesenchymal stem cell.

33. The exosome composition of claim 32, wherein the fetal mesenchymal stem cell is a human cell.

34. A pharmaceutical composition comprising a conditioned medium preparation comprising medium conditioned by a fetal mesenchymal stem cell, and a pharmaceutically acceptable carrier.

35. A pharmaceutical composition comprising an exosome isolated from medium conditioned by a fetal mesenchymal stem cell, and a pharmaceutically acceptable carrier.
